(19) United States
(12) Patent Application Publication
(10) Pub. No.: US 2007/0123486 A1
(43) Pub. Date: May 31, 2007

(54) Composition for coordinated VEGF and PDGF expression, and methods of use

(76) Inventors: Andrea Banfi, Basel (CH); Helen M. Blau, Menlo Park, CA (US); Georges J. Von Degenfeld, Wuppertal (DE)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/600,666

(22) Filed: Nov. 15, 2006

Related U.S. Application Data
(60) Provisional application No. 60/737,255, filed on Nov. 15, 2005.

(51) Int. Cl. A61K 48/00; C07H 21/04; C12P 21/06; C07K 14/475

(52) U.S. Cl. 514/44; 435/69.1; 435/320.1; 435/325; 530/399; 536/23.5

(57) ABSTRACT

Compositions for co-expression of VEGF and PDGF at a desired ratio, and their methods of use, are provided.

### CROSS-REFERENCE TO RELATED APPLICATION

This application claims priority benefit of U.S. provisional application Ser. No. 60/737,255, filed Nov. 15, 2005, which application is incorporated herein by reference in its entirety.

### BACKGROUND

Angiogenesis has been the focus of intense interest since these processes can be exploited to therapeutic advantage. Stimulation of angiogenesis can, for example, aid in the healing of wounds, the vascularizing of skin grafts, tissue engineering, and the enhancement of collateral circulation where there has been vascular occlusion or stenosis (e.g., to develop a “biobypass” around an obstruction due to coronary, carotid, or peripheral arterial occlusion disease). Angiogenesis is also an important component of stroke, head injury, cerebral vascular malformation development and brain tumor growth.

Vascular endothelial growth factor (VEGF) has been the focus of much attention as a factor to promote angiogenesis. However, many investigators have shown that an excess of VEGF can lead to hemangiomas (vascular tumors). Regulating levels of VEGF in a therapeutically efficacious manner can be difficult using currently available delivery vectors. Furthermore, the microenvironmental dose of VEGF is important. (see, e.g., Ozawa et al. J. Clin Invest. 113:516-527 (2004); Stiver et al. J. Neuropathol. Exp. Neurol. 63:841-855, (2004); Dor et al. Ann NY Acad. Sci. 995:208-216 (2003)).

### SUMMARY OF THE INVENTION

Compositions for coordinated expression of VEGF and PDGF, and their methods of use, are provided.

### BRIEF DESCRIPTION OF THE DRAWINGS

The patent or application file contains at least one drawing executed in color. Copies of this patent or application publication with color drawing(s) will be provided by the U.S. Patent and Trademark Office upon request and payment of necessary fee. It is emphasized that, according to common practice, the various features of the drawings are not necessarily to-scale, and dimensions of the various features are arbitrarily expanded or reduced for clarity. Included in the drawings are the following figures:

FIG. 1 illustrates the aberrant angiogenesis by uncoordinated delivery of VEGF and PDGF-BB. Panel A: a map of the retroviral constructs used to generate myoblast populations expressing murine VEGF₁₆₄ (pMFG-V) (“V”) or human PDGF-BB (pAMFG-P) (“P”). Panels (B-K) whole-mount staining of blood vessels in ear muscles implanted with control myoblasts (Panel B), (n=7), or myoblasts expressing PDGF-BB (Panel C), (P cells, n=7), VEGF₁₆₄ (Panel D), (V cells, n=7), a mixture of the 2 populations in equal proportion (Panels E-G), (50:50, n=7), or at a ratio of V:P cells 1:50 (Panels H-I), (V1, n=4) or 50:20 (Panels J and K), (P20, n=4). White arrows in Panel F indicate the aberrant structures right next to an area of normal angiogenesis. Size bars=25 μm in all panels.

FIG. 2 illustrates normal angiogenesis by microenvironmental co-localization of VEGF and PDGF-BB with coordiantely regulated of expression (transcription and translation). Panel A: Generation of myoblasts co-expressing VEGF and PDGF-BB at random relative levels by sequential transduction with two separate retroviral constructs (VP cells). Panels (B-D): Whole-mount staining of blood vessels in ear muscles implanted with myoblasts expressing VEGF alone (Panel B), or with VP cells (Panels C-D); n=5. Panel E: Map of the VIP retroviral construct to achieve co-expression of VEGF and PDGF-BB at matched microenvironmental levels around each transduced cell. Panels (F-H): Whole-mount staining of blood vessels in ear muscles implanted with VIP_{high} myoblasts: white arrows in h indicate sprouting capillaries; n=10. Panels (I-J): Quantification of the amount of angiogenesis (Panel I) and the distribution of vessel diameters (Panel J) in areas implanted with control cells (CD8), PDGF-BB (P), VEGF (V) or VIP_{high} myoblasts (VIP). VLD=vessel length density, expressed as mm of vessel length/mm² of area of effect. Values in i are given as the mean±s.e.m. of each condition (n=4-5). ***, p<0.001. Size bars=25 μm in all panels.

FIG. 3 illustrates the proper maturation of vessels induced by coordinately regulated expression (transcription and translation) of VEGF and PDGF-BB. Panel (A-E): Immunostaining of vessels induced by implantation of control CD8 cells (Panel A) or VEGF (Panel B), PDGF-BB (Panel C) or VIP_{high} myoblasts (Panel D) and (Panel E) with antibodies against CD31 (green, endothelium), NG2 (red, pericytes) and α-SMA (blue in a-d, smooth muscle) or laminin (blue in e, basement membrane). White arrows in Panels A and E point to pericytes displaying the typical branched processes. Asterisks in Panel B indicate the lumen of a bulbous angiomatous vessel devoid of pericytes and covered with smooth muscle cells. Size bars=25 μm in all panels.

FIG. 4 illustrates that PDGF-BB co-expression does not adversely affect vascular induction by VEGF and yields non-leaky vessels after remodeling. Panels (A-D): Whole-mount staining of blood vessels at the initial stage of angiogenic induction 4 days after implantation with control CD8 cells (Panel A), or PDGF-BB (Panel B), VEGF (Panel C) or VIP_{high} myoblasts (Panel D); n=4. Panel E: Quantification of vascular leakage induced by control cells (CD8) or myoblasts expressing PDGF-BB (P), VEGF (V) or both (VIP) at 4 days, 1 and 2 weeks, expressed as ng of extravasated Evans blue/mg of tissue weight (±s.e.m., n=5). Panel F: Normalization of vascular leakage by the amount of induced vasculature 2 weeks after implantation of the same populations, expressed as ng of extravasated Evans blue/mm² of total vessel surface (±s.e.m., n=5). *=p<0.05,**=p<0.01. Size bars=25 μm in all panels.

FIG. 5A-5E illustrates the improvement of blood perfusion and collateral formation in hindlimb ischemia. FIG. 5A: Immunostaining of treated ischemic muscles with antibodies against CD31 (red, endothelium), NG2 (green, pericytes) or α-SMA (blue, smooth muscle). FIG. 5B: Quantification of the angiogenic response in the ischemic treated legs (blue bars) and the contralateral non-ischemic legs (white bars). Vessel length density is expressed as μm of vessel length/muscle fiber in the area of effect±s.e.m. (n=6-9). FIG. 5C: Blood flow in the ischemic limbs, expressed as a percentage of the contralateral non-ischemic leg±s.e.m. (n=5-7). The dotted line represents non-ischemic flow (100%). FIG. 5D: Number of collaterals in the adductor muscle group of ischemic treated legs±s.e.m. (n=3). FIG. 5E: Damaged area in ischemic muscles of treated limbs, expressed as a percentage of the total muscle surface on histological sections±s.e.m. (n=5-6). *, p<0.05, **, p<0.01.

FIG. 6 illustrates that PDGF-BB modulates the threshold between normal and aberrant angiogenesis. Panel (A-H): Whole-mount staining of blood vessels in ear muscles implanted with myoblast clones expressing a high (Panel A) or low (Panel E) VEGF level with concomitant PDGF-BB over-expression (Panel C) or blockade (Panels A and D). Control cells expressing only sPDGFRβ (Panel F), CD8 (Panel G) or PDGF-BB (Panel H) did not cause any angiogenic effect. Size bars=25 μm in all panels; n=5, except for Panel F (n=2).

FIG. 7 illustrates the co-expression of VEGF-A and PDGF-BB by clonal myoblast populations. The stably transduced individual clones co-express mVEGF₁₆₄ and hPDGF-BB from the VIP construct. The presence of both factors in the supernatants were quantified by ELISA resulting in a range of expression from 3 to 200 ng/10⁶ cells/day of VEGF and a relative ratio of 0.46, with a correlation coeffficient R²=0.93.

Definitions

By “coordinated expression”, “coordinately regulated expression” or “co-regulated expression” as used interchangeably herein in reference to a polynucleotide encoding both PDGF and VEGF is meant that the polynucleotide (e.g., expression cassette, construct) provides for production of a desired ratio of PDGF:VEGF as a result of regulation at the translational level (e.g., as in the bicistronic expression cassettes described herein), at the transcriptional level (e.g., as in the dual expression cassettes described herein), or both (e.g., as in the dual expression cassettes in the sense that translation levels are dependent upon transcription levels). The desired ratio of PDGF:VEGF as produced from such co-regulated expression constructs can be selected and fixed for a given construct through selection of transcriptional and translational regulatory elements, e.g., translation initiation signal, promoters, and the like.

By “coordinated delivery of PDGF and VEGF” is meant delivery of PDGF and VEGF from a recombinant cell as a result of expression of a construct in the recombinant cell that provides for coordinated expression of PDGF and VEGF. In contrast “uncoordinated delivery” of PDGF and VEGF indicates that PDGF and VEGF are expressed from separate constructs present in different cells.

As used herein “angiogenesis” refers generally to a process by which new blood vessels are formed from extant capillaries, while “vasculogenesis” refers generally to a process involving growth of vessels deriving from endothelial progenitor cells, and can involve recruitment and differentiation of mesenchymal cells into angioblasts, which then differentiation into endothelial cells and form de novo vessels. Reference to “angiogenesis” and to “vasculogenesis” in the specification is not mean to imply that the compositions are useful for only one of such processes, but rather is only exemplary of the use to which the compositions can be applied.

By “non-aberrant angiogenesis” and “non-aberrant vasculogenesis” is meant angiogenesis or vasculogenesis, respectively, with little or not detectable formation of defective vascular structures which differ in one or more of structure, function, size and/or shape from normal capillaries, arteries or veins, which defective vascular structures can include but are not limited to glomeruloid, globular, hemangiomas and/or hemangioma-like structures.

“Treating” or “treatment” of a condition or disease includes: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms.

A “therapeutically effective amount” or “efficacious amount” means the amount of a compound that, when administered to a mammal or other subject for treating a disease, is sufficient to effect such treatment for the disease. The “therapeutically effective amount” will vary depending on the compound, the disease and its severity and the age, weight, etc., of the subject to be treated.

By “operably linked”, “operably positioned” or “operably joined” in the context of nucleic acid means that a DNA sequence and a regulatory sequence(s) are connected in such a way as to permit gene expression when the appropriate molecules (e.g., transcriptional activator proteins) are bound to the regulatory sequence(s). “Operably linked”, “operably positioned” or “operably joined” in the context of a polypeptide means that the portions of the polypeptide are present so as to provide for a polypeptide having a desired biological activity (e.g., promotion of transcriptional activation).

By “heterologous” refers to the situation where a first material is associated with a second material, where the first and second materials are not associated in this manner in nature.

The term “cDNA” as used herein is intended to include all nucleic acids that share the arrangement of sequence elements found in native mature mRNA species, where sequence elements are exons (e.g., sequences encoding open reading frames of the encoded polypeptide) and 3′ and 5′ non-coding regions. Normally mRNA species have contiguous exons, with the intervening introns removed by nuclear RNA splicing, to create a continuous open reading frame encoding the polypeptide of interest.

As used herein the term “isolated” is meant to describe a compound of interest (e.g., either a polynucleotide or a polypeptide) that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound (e.g., either a polynucleotide or a polypeptide) that is removed from its natural environment and is at least 60% free, preferably 75% free, and most preferably 90% free from other components with which it is naturally associated.

By “transformation” or “transfection” (as well as “transformed” or “transfected”) is meant a permanent or transient genetic change induced in a cell following incorporation of new nucleic acid (e.g., DNA or RNA exogenous to the cell). Genetic change can be accomplished either by incorporation of the new nucleic acid into the genome of the host cell, or by transient or stable maintenance of the new DNA as an episomal element.

By “recombinant cell” is meant a cell into which (or into an ancestor of which) has been introduced, by means of recombinant DNA techniques, a DNA molecule encoding a protein of interest. “Recombinant cell” thus refers to such a recombinantly modified cell, or progeny thereof which retain such genetic modification and/or produce a gene product encoded by the genetic modification.

By “construct” or “vector”, used interchangeably herein, is meant a recombinant nucleic acid, generally recombinant DNA, that has been generated for the purpose of the expression of a specific nucleotide sequence(s), or is to be used in the construction of other recombinant nucleotide sequences. An “expression cassette” refers to a polynucleotide composed of elements that provide for, or upon operable insertion into a vector or host cell provide for, expression of gene product( )s encoded by one or more polynucleotides contained in the expression cassette (e.g., VEGF and PDGF.

The term “transgene” is used herein to describe genetic material which has been or is about to be artificially inserted, either transiently or permanently, into a cell of an animal, particularly a mammalian cell, e.g., in a culture or in a living animal.

By “target cell” or “host cell” is meant any cell selected for genetic modification, e.g., contain a bicistronic expression cassette or dual expression cassette vector described herein. Such cells can include cells in which expression may be desired (e.g., immortalized mammalian cell lines, primary cultured cells (e.g., mammalian cells, including human cells), and the like) as well as cells that can provide for replication of the vector which may or may not be accompanied by expression (e.g., bacterial host cells, yeast cells, insect cells, including packaging cells for production of virus containing the vector, and the like). The use of “target cell” throughout the specification is for convenience only, and is not meant to imply that, for example, accomplishing introduction of a nucleic acid of interest into a cell, particularly in vivo (e.g., in situ), which may optionally exploit the use of targeting techniques (e.g., targeting molecules that preferentially direct the material to be introduced to a particular cell or cell type).

The terms “subject” and “patient” mean a member or members of any mammalian or non-mammalian species that may have a need for the methods, compositions and treatments described herein. Subjects and patients thus include, without limitation, primate (including humans), canine, feline, ungulate (e.g., equine, bovine, swine (e.g., pig)), avian, and other subjects. Humans and non-human animals having commercial importance (e.g., livestock and domesticated animals) are of particular interest.

“Mammal” means a member or members of any mammalian species, and includes, by way of example, canines; felines; equines; bovines; ovines; rodentia, etc. and primates, particularly humans. Non-human animal models, particularly mammals, e.g. primate, murine, lagomorpha, etc. may be used for experimental investigations.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable carrier. The specifications for the novel unit dosage forms of the present invention depend on, for example, the effect to be achieved in the host.

A “pharmaceutically acceptable carrier,” means a substance useful in preparing a pharmaceutical composition that are generally safe, non-toxic and neither biologically nor otherwise undesirable, and include such acceptable for veterinary use as well as human pharmaceutical use. “A pharmaceutically acceptable carrier” as used in the specification and claims includes both one and more than one such excipients.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade).

Before additional disclosure of embodiments of the present invention is provided, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described.

All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a vector” includes a plurality of such vectors and reference to “the protein” includes reference to one or more proteins and equivalents thereof known to those skilled in the art, and so forth.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing-herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

Compositions for coordinated expression of VEGF and PDGF, and their methods of use, are provided.

Described herein are recombinant polynucleotides, including isolated recombinant polynucleotides, which encode expression cassettes which provide for expression of VEGF and PDGF from a single construct. In general, such expression cassettes contain a first polynucleotide encoding a vascular endothelial growth factor (VEGF) polypeptide or a platelet-derived growth factor (PDGF) polypeptide; a second polynucleotide encoding a second polypeptide, wherein when the first polynucleotide encodes a VEGF polypeptide, the second polypeptide is a PDGF polypeptide and when the first polynucleotide encodes a PDGF polypeptide the second polypeptide is a VEGF polypeptide; and either a translation initiation signal (e.g., an internal ribosome entry site (IRES)) (in bicistronic expression cassettes) or a promoter (in dual expression cassettes), operably positioned between the first and second polynucleotides. The recombinant polynucleotides can further include a promoter at its 5′ end, wherein the promoter is operably positioned to provide for transcription of the first polynucleotide. In bicistronic expression cassettes, the 5′ promoter provides for expression of the first polynucleotide, the second polynucleotide and translation initiation signal (e.g., IRES) as a single RNA molecule. In embodiments of particular interest, the first polynucleotide encodes VEGF and the second polynucleotide encodes PDGF. Such recombinant polynucleotides can be inserted into a vector (e.g., as a expression cassette) to provide an expression of a bicistronic transcript or for expression of PDGF and VEGF as separate mRNAs.

The present description also provides expression vectors containing a bicistronic expression cassette or a dual expression cassette as described above.

The present disclosure also provides recombinant host cells which contain the recombinant polynucleotides and vectors set out above, as well as methods of making such recombinant host cells by introduction of such polynucleotides and/or vectors into a host cell (e.g., to provide for at least transient, usually stable, maintenance of the exogenous nucleic acid in the cell).

The present disclosure also provides methods, of administering a PDGF polypeptide and a VEGF polypeptide to a subject, which can be accomplished by administering to a subject a recombinant polynucleotide or vector as described herein, usually a vector, in an amount effective to provide production of PDGF and VEGF in the subject (e.g., as a result of introduction of the vector into a target cell in the subject). In one embodiment, the vector or recombinant polynucleotide is contained in a recombinant host cell, which cell is adapted for expression to provide for VEGF and PDGF production in the host.

The present disclosure also provides methods for stimulating angiogenesis in a subject having a condition that is treatable by stimulating angiogenesis, which method includes administering to a mammal an effective amount of a recombinant polynucleotide or bicistronic vector as described herein, wherein said administering of the vector provides for production of VEGF and PDGF to effect stimulation of angiogenesis in the subject. In one embodiment, the vector or recombinant polynucleotide is contained in a recombinant host cell, which cell is adapted for expression to provide for VEGF and PDGF production in the host.

Various aspects and elements of the invention will now be described.

Compositions for Delivery of VEGF and PDGF

Compositions for delivery of VEGF and PDGF include polynucleotides comprising an expression cassette that provides for production of VEGF and PDGF from a single polynucleotide, which can be bicistronic expression cassettes that provide for transcription of VEGF- and PDGF-encoding DNA as a single mRNA or dual expression cassettes that provide for transcription of VEGF- and PDGF-encoding DNA as separate mRNAs. Compositions for delivery of VEGF and PDGF also include recombinant host cells genetically modified to express the expression cassettes encoding PDGF and VEGF.

In some embodiments, the composition does not contain angiopoietin (e.g., Ang-1, Ang-2), Tie-2, transforming growth factor beta (TGF-β), TGF-β receptor (e.g., TGF-β2 receptor) endoglin, Smad5, VE-Cadherin, ephrinβ2, Bmx tyrosine kinase, MCP-1, fibroblast growth factor (e.g., acidic FGF (aFGF), basic FGF (bFGF)), leptin and/or hepatocyte growth factor (HGF). With respect to recombinant host cells, the host cell is not genetically modified to produce an exogenous polypeptide of angiopoietin (e.g., Ang-1, Ang-2), Tie-2, transforming growth factor beta (TGF-β), TGF-β receptor (e.g., TGF-β receptor) endoglin, Smad5, VE-Cadherin, ephrinβ2, Bmx tyrosine kinase, MCP-1, fibroblast growth factor (e.g., acidic FGF (aFGF), basic FGF (bFGF)), leptin and/or hepatocyte growth factor (HGF). By “exogenous” is meant a polypeptide or polynucleotide that is not native to a host cell.

Expression Cassettes And Vectors

Vectors of interest are those that provide for a desired ratio production of PDGF:VEGF polypeptides in a cell, where, once selected, the desired ratio for a given construct can be fixed (e.g., to provide for a substantially consistent ratio of PGDF:VEGF production by the cell in which the construct is expressed). Accordingly, the vectors disclosed herein are those in which the vector elements are selected to provide for the desired ratio of PDGF:VEGF polypeptide in a cell expressing the construct. Expression cassettes to provide for PDGF and VEGF production are exemplified by bicistronic expression cassettes and dual expression cassette vectors. The expression cassettes include those the provide for single-stranded polynucleotide which contains the coding sequence and regulatory elements of the formulae below (e.g., as in bicistronic expression cassettes and certain embodiments of the dual expression cassette (e.g., where the cassettes involve two promoters which provide for transcription from the same strand of the nucleic acid), as well as those that provide for expression of VEGF and PDGF from a single double-stranded nucleic acid, but which involve expression from different strands of the nucleic acid (e.g., as in the dual expression vectors having “opposite facing” promoters with respect to direction of transcription, which are positioned on complementary strands of the expression cassette within a construct).

Expression cassettes for coordinated co-expression of PDGF and VEGF can be described by a polynucleotide sequence represented by the formula:
P₁-A-Z-B

where

P₁, when present, is a first promoter operably linked to provide for expression of DNA positioned 3′ of P₁ (e.g., to provide for production of an mRNA encoding A or A-Z-B) and, when P₁ is absent, expression can be provided following insertion of the expression cassette into a vector which contains a promoter or following integration into a genomic sequence which provides for an operably linked promoter;

“A” is a first polynucleotide comprising a first coding sequence for either a VEGF polypeptide or a PDGF polypeptide;

“B” is a second polynucleotide comprising a second coding sequence wherein, when A encodes a VEGF polypeptide B encodes a PDGF polypeptide or, when A encodes a PDGF polypeptide B encodes a VEGF polypeptide; and

“Z” is a polynucleotide comprising
a translation initiation signal “T” (wherein T can be, e.g., an IRES (internal ribosome entry site) or a non-IRES translation initiation signal (e.g., a polyribosomal slippage sequence)), which is operably positioned to facilitate translation of a polypeptide encoded by B from a single mRNA encoding A, T, and B;
a promoter “P₂”, wherein P₂ is operably positioned to facilitate transcription of an mRNA encoded by B, and wherein P₁, when present facilitates transcription of an mRNA encoded by A (such that A and B are transcribed as separate mRNAs); or
a promoter “P₃” and a promoter “P₄” wherein P₃ is operably positioned to provide for transcription of an mRNA encoded by the coding sequence of A and P₄ is operably positioned to provide for transcription of an mRNA encoded by the coding sequence of B. In this latter embodiment, P₃, P₄, and the coding sequences to which they are operably linked (e.g., A-P₃ (which may also be represented by P₃-A) and P₄-B are provided on opposite, complementary strands of the double-stranded polynucleotide.

Specific exemplary vectors can be represented by the formulae P₁-VEGF-Z-PDGF; P₁-PDGF-Z-VEGF; P₁-VEGF-T-PDGF; P₁-PDGF-T-VEGF; P₁-VEGF-P₂-PDGF; P₁-PDGF-P₂-VEGF; PDGF<-P₃ P₄->VEGF; and VEGF<-P₃ P₄->PDGF, where the direction of the arrows in the latter two constructs denotes the “direction” of transcription from complementary strands in a double-stranded nucleic acid. PDGF can be a PDGFb.

Bicistronic Vectors

The compositions of the invention include a bicistronic vector that provides for expression of both VEGF and PDGF from a single vector and as a single mRNA. The term “bicistronic” refers to a polynucleotide construct that provides for translation of two separate or unrelated cistrons from a single transcriptional unit. In the present invention, the vector is composed of first and second polynucleotides, which provide coding sequences for VEGF and PDGF as described below, and transcription initiation signal (e.g., an IRES) operably positioned between the first and second coding sequences. The first coding sequence, the transcription initiation signal (e.g., IRES), and the second coding sequences are expressed as a single transcription unit (single mRNA), and translation can be initiated for the mRNA corresponding to both the first and second coding sequences from this single mRNA. In this manner, the vector can provide for expression of VEGF and PDGF in a desired ratio, which can be fixed for a given host cell.

In general, bicistronic expression cassettes disclosed herein comprise a polynucleotide encoding an expression cassette having a sequence represented by the formula:
A-T-B
“A” is a first polynucleotide comprising a coding sequence for either VEGF or for PDGF; “T” is a translation initiation signal (e.g., an IRES (internal ribosome entry site) or polyribosomal slippage sequence), which is operably positioned 5′ of “B”; and “B” is a second polynucleotide comprising a second coding sequence which, where A is VEGF, encodes PDGF or, where A is PDGF, encodes VEGF. Specific vectors can be represented by the formulae VEGF-T-PDGF or PDGF-T-VEGF. The expression cassette can optionally include a promoter P₁ operably positioned 5′ of A.

Other bicistronic expression cassettes contemplated by the disclosure herein include those comprising a polynucleotide having a sequence represented by the formula:
A-IRES-B
where “A” is a first cistron comprising a coding sequence for either VEGF or for PDGF; “IRES” refers to an internal ribosome entry site, which is operably positioned 5′ of “B”; and “B” is a second cistron comprising a second coding sequence which, where A is VEGF, encodes PDGF or, where A is PDGF, encodes VEGF. The bicistronic expression cassettes described herein can also be represented by the formulae:
VEGF-IRES-PDGF
or
PDGF-IRES-VEGF
where “VEGF” refers to a polynucleotide encoding a VEGF polypeptide; “IRES” refers to an internal ribosome entry site operably linked to the coding sequence positioned 3′ of the IRES sequence; and “PDGF” refers to a polynucleotide encoding a PDGF polypeptide. The expression cassette can optionally include a promoter P₁ operably positioned at the 5′ end of the expression cassette. PDGF can be a PDGFb. In other embodiments, PDGF is not PDGFa.

In general, while two cistrons in a bicistronic expression cassette are fixed with respect to copy number for expression, the elements of the cassette can be selected so as to provide for quantitatively equal or unequal production of the encoded gene products. For example, as discussed in the Examples below, the elements of the cassette can be selected to that the gene product encoded in the cistron in the second position (i.e., 3′ of T, e.g., 3′ of the IRES) is produced at lower levels compared to the gene product of the first cistron (i.e., 5′ of T, e.g., 5′ of the IRES). This phenomenon can be exploited to provide for higher levels of one of a VEGF relative to a PDGF, or vice versa.

For example, bicistronic expression cassettes and vectors can have elements (e.g., promoter, translation initiation signal, etc.) that provide for a level of PDGF production that is about 5%, about 10%, about 15%, about 20%, about 25%, about 30%, about 35%, about 40%, about 45%, about 50% about 55%, about 65%, about 70%, about 75%, about 80%, about 85%, about 90%, about 95%, about 100%, about 105%, about 110%, about 115%, about 120%, about 125%, about 130% or more of a VEGF expression level on a molar basis, including ranges of from about 5% to 130%, about 10% to 90%, about 20% to 80%, about 25% to 65%, about 30% to 50%, and the like. Bicistronic expression cassettes and vectors can have elements that provide for a ratio of PDGF:VEGF polypeptide of about 1:20, 1:15, 1:10, 1:5, 1:4, 1:3, 1:2, 1:1, 2:1, 3:1, 4:1, 5:1, 6:1 and the like. In some embodiments, the ratio of PDGF:VEGF is less than 3:2. Bicistronic expression cassettes and vectors can have elements that provide for VEGF polypeptide production in amounts ranging from about 0.1 to 100 ng/10⁶ cells/day or more, from about 0.5 to 90 ng/10⁶ cells/day or more, from about 0.8 to 75 ng/10⁶ cells/day or more, from about 1 ng to 65 ng/10⁶ cells/day or more, from about 1 ng to 50 ng/10⁶ cells/day or more. Bicistronic expression cassettes and vectors may have elements that provide for PDGF-BB polypeptide production from about 0.8 ng to 64 ng/10⁶ cells/day.

Expression of the bicistronic expression cassette can be provided by any suitable promoter, which is positioned 5′ of the first coding sequence of the vector (“P₁”). Of particular interest are promoters operable in a eukaryotic cell, e.g., a mammalian cell, more particularly a human cell. It should be noted that the inventors have found that viral infection or cDNA transfection efficiency, transcriptional activity, or random integration site of the vector did not alter efficacy. Thus, the expression cassette can also be provided so as to provide for integration at a desired site in a genome so as to provide for expression of the bicistronic vector from a promoter endogenous to a target cell.

Dual Expression Cassette Vectors

A dual expression cassette vector provides PDGF- and VEGF-encoding DNA on a single vector which are under control of separate promoters. “Dual expression cassettes” refer to polynucleotides that provide for production of two mRNAs, each encoding one of VEGF and PDGF. Promoters operably linked to the two coding sequences are selected to provide for a desired ratio of expression, and thus a desired ratio of polypeptide, in the cell. In general, the a dual expression cassette is composed of first and second polynucleotides, which provide coding sequences for VEGF and PDGF as described below, and separate promoters operably linked to each of the first and second polynucleotide, where the promoter positioned at the 5′ end of the expression cassette can be provided as part of the expression cassette, upon insertion into a vector, or upon integration into a genomic sequence of a host cell. Selection of the two promoters can provide for expression of VEGF and PDGF in a desired “fixed” ratio of VEGF to PDGF in each host cell.

In general, dual expression cassettes comprise a polynucleotide encoding an expression cassette having a sequence represented by the formula:
A-Pₓ-B

“A” is a first polynucleotide comprising a coding sequence for either VEGF or for PDGF;

“B” is a second polynucleotide comprising a second coding sequence which, where A is VEGF, encodes PDGF or, where A is PDGF, encodes VEGF; and

“Pₓ” is a polynucleotide comprises
a promoter “P₂” operably positioned to facilitate transcription of an mRNA encoded by B, where a promoter operably positioned adjacent the coding sequence of A facilitates transcription of an mRNA encoded by A (such that A and B are transcribed as separate mRNAs and in the same transcriptional “direction”; or
a promoter “P₃” and a promoter “P₄” wherein P₃ is operably positioned to provide for transcription of an mRNA encoded by the coding sequence of A and P₄ is operably positioned to provide for transcription of an mRNA encoded by the coding sequence of B. In this latter embodiment, P₃, P₄, and the coding sequences to which they are operably linked (e.g., A-P₃ (which may also be represented by P₃-A) and P₄-B are provided on opposite, complementary strands of the double-stranded polynucleotide.

Specific vectors can be represented by the formulae VEGF-P₂-PDGF or PDGF-P₂-VEGF. The expression cassette can optionally include a promoter P₁ operably positioned 5′ of A. Further exemplary vectors include those encoding PDGF<-P₃ P₄->VEGF; and VEGF<-P₃ P₄->PDGF, where the direction of the arrows in the latter two constructs denotes the “direction” of transcription from complementary strands in a double-stranded nucleic acid.

In general, a desired ratio PDGF:VEGF production is provided by selecting elements of the vector can be selected so as to provide for quantitatively equal or unequal production of the encoded gene products, e.g., by selecting the promoters so as to provide desired relative expression levels. For example, as discussed in the Examples below, the elements of the vector can be selected to that the gene product encoded in the coding in the second position (i.e., 3′ of P₁) is produced at lower levels compared to the gene product of the first polynucleotide (i.e., 5′ of P₁). This phenomenon can be exploited to provide for higher levels of one of a VEGF relative to a PDGF, or vice versa.

For example, dual expression cassettes and vectors can have elements (e.g., promoter, translation initiation signal, etc.) that provide for a level of PDGF production that is about 5%, about 10%, about 15%, about 20%, about 25%, about 30%, about 35%, about 40%, about 45%, about 50% about 55%, about 65%, about 70%, about 75%, about 80%, about 85%, about 90%, about 95%, about 100%, about 105%, about 110%, about 115%, about 120%, about 125%, about 130% or more of a VEGF expression level on a molar basis, including ranges of from about 5% to 130%, about 10% to 90%, about 20% to 80%, about 25% to 65%, about 30% to 50%, and the like. Dual expression cassettes and vectors can have elements that provide for a ratio of PDGF:VEGF polypeptide of 1:20, 1:15, 1:10, 1:5, 1:4, 1:3, 1:2, 1:1, 2:1, 3:1, 4:1, 5:1, 6:1, and the like. In some embodiments, the ratio of PDGF:VEGF is not 3:2, i.e., is greater than or less than 3:2. Dual expression cassettes and vectors can have elements that provide for VEGF polypeptide production in amounts ranging from about 0.1 to 100 ng/10⁶ cells/day or more, from about 0.5 to 90 ng/10⁶ cells/day or more, from about 0.8 to 75 ng/10⁶ cells/day or more, from about 1 ng to 65 ng/10⁶ cells/day or more, from about 1 ng to 50 ng/10⁶ cells/day or more. Bicistronic expression cassettes and vectors may have elements that provide for PDGF-BB polypeptide production from about 0.8 ng to 64 ng/10⁶ cells/day,

The promoters of the dual expression cassette can be selected from any suitable promoter, with promoters operable in a eukaryotic cell, e.g., a mammalian cell, more particularly a human cell, being of interest. The expression cassette can include a promoter positioned 5′ of the first coding sequence of the vector (“P₁”). It should be noted that the inventors have found that viral infection or cDNA transfection efficiency, transcriptional activity, or random integration site of the vector did not alter efficacy. Thus, the vectors can also be provided so as to provide for integration at a desired site in a genome so as to provide for expression of the coding sequence “A” from a promoter endogenous to a target cell.

In dual expression constructs, the promoters of the construct (e.g., P₁ and P₂, or P₃ and P₄) can be the same (e.g., to provide for expression, and thus polypeptide production, at a 1:1 ratio of PDGF:VEGF) or can be different (e.g., to provide for different ratios of expression, and thus different ratios of PDGF:VEGF produced in a cell). Examples of promoters include constitutive promoters, strong promoters, weak promoters, inducible promoters, and tissue-specific promoters (e.g., muscle-specific, endothelial cell-specific, etc.). In one embodiment, the two promoters are selected as having different transcriptional “strengths” relative to one another so as to provide different transcription levels of a PDGF-encoding DNA and a VEGF-encoding DNA and thus vary the ratio of PDGF:VEGF production. For example, the promoters can be selected so that a first promoter is a constitutive strong promoter (e.g., an EF2a (Elongation factor 2a) promoter) and a second promoter is a weak promoter (e.g., a thymidine kinase (TK) promoter). Further exemplary promoters are discussed below.

Vector Production

Production of vectors can be accomplished using standard techniques of molecular biology, such as described in numerous standard protocol texts, including e.g., Current Protocols in Molecular Biology, (F. M. Ausubel, et al., Eds. 1987, and updates. The vector can be based on any suitable backbone, such as a viral vector, e.g., a retroviral vector, adenoviral vector, AAV vector. Suitable vectors are well known in the art, and can be readily adapted for use in the compositions and methods described herein.

VEGF

“Vascular endothelial growth factor polypeptide” or “VEGF polypeptide” as used herein encompasses a native VEGF polypeptide (including splice variants), or a functional fragment or variant of a native VEGF polypeptide (e.g., VEGF₁₆₄) having activity in promoting angiogenesis, particularly when co-administered with PDGF to provide for promoting non-aberrant angiogenesis. Usually the VEGF is a human VEGF, with native human VEGF and angiogenesis-promoting fragments thereof being of particular interest. In the context of expression cassettes, constructs and vectors, “VEGF” is meant to refer to a coding sequence encoding a VEGF monomer, where expression and translation of VEGF monomers can provide for production of VEGF dimers. It will be clear to the ordinarily skilled artisan that reference to a vector that expresses “VEGF”, where the vector may include only one coding sequence for VEGF, is meant to indicate that a VEGF dimer is assembled following transcription and translation of the VEGF monomer.

VEGF polypeptides, and accordingly VEGF-encoding polynucleotides, useful in the embodiments described herein include VEGF-1 (also known as VEGF-A), VEGF-2 (VEGF-C), VEGF-3 (VEGF-B), VEGF-D, VEGF-E, and the like. VEGF-1 isoforms useful herein can include, for example, 121, 138, 162, 165, 182, 189, and 206 amino acids. These isoforms are identified, respectively, as VEGF-121, VEGF-165, VEGF-162, VEGF-182, VEGF-189, and VEGF-206. Polynucleotides encoding VEGF polypeptides, as well as fragments and variants thereof having activity in promoting angiogenesis, are well known in the art, and can be readily adapted for use in the constructs and methods described herein. See, e.g., U.S. Pat. No. 6,020,473; U.S. Pat. No. 6,395,707: U.S. Pat. No. 6,057,428; U.S. Pat. No. 6,485,942; U.S. Pat. No. 6,750,044; U.S. Pat. No. 6,057,428; US 20050215466; US 20050020522; US 20050158281; US 20040161412; each of which references is incorporated herein by in its entirety.

PDGF

“Platelet derived growth factor polypeptide” or “PDGF polypeptide” as used herein encompasses a PDGF B chain (PDGFb) polypeptide, or a functional fragment or variant of a native PDGFb having activity in modulating angiogenesis, particularly when co-administered with a VEGF polypeptide as described herein to provide for stimulating non-aberrant angiogenesis. “Platelet derived growth factor polypeptide-b” or “PDGFb polypeptide”, and accordingly PDGF-encoding polynucleotides, useful in the embodiments described herein include a PDGF B chain (PDGFb) polypeptide, or a functional fragment or variant of a native PDGFb having activity in modulating angiogenesis, particularly when co-administered with a VEGF polypeptide as described herein. Usually the PDGF is a human PDGF, with human PDGFb being of particular interest. The vector can thus provide for production of PDGF-BB dimers of PDGF following expression of the encoded polypeptide. In the context of vectors, “PDGF” is meant to refer to a coding sequence encoding a PDGF monomer, where expression and translation of PDGF monomers can provide for production of PDGF dimers. It will be clear to the ordinarily skilled artisan that reference to a vector that expresses “PDGF-BB”, where the vector may include only one coding sequence for PDGF-B, is meant to indicate that the BB dimer is assembled following expression and translation of B monomer. In general, the PDGF encoded by the construct is not PDGFa.

Biologically active fragments, analogs, and derivatives thereof, and polynucleotide encoding such, are well known in the art and can be readily adapted for use in the constructs and methods described herein. See, e.g., U.S. Pat. No. 5,187,263; Waterfield et al., Nature 304:35-39 (1983); Wang et al., J. Biol. Chem. 259: 10645-48 (1984), Antoniades et al., Biochem. Pharm. 33: 2833-38 (1984); and Westermark et al., Proc. Natl. Acad. Sci USA 83:7197-7200 (1986); U.S. Pat. No. 5,219,759, which references are incorporated herein in their entireties.

Translation Initiation Signal

“Translation initiation signal” or “TIS” refers to a polynucleotide having a sequence encoding an mRNA to which ribosomes bind and provide for translation of a coding sequence positioned 3′ of the translation initiation signal. Examples of TIS include IRES′ and non-IRES translation initiation signals.

IRES

The term “internal ribosomal entry site” or “IRES” refers to a viral, cellular, or synthetic (e.g., a recombinant) nucleotide sequence which allows for initiation of translation of an mRNA at a site internal to (e.g., at a site 3′ of) a first coding region within the same mRNA or at a site 3′ of the 5′ end of the mRNA, to provide for translation of an operably linked coding region located downstream of (i.e., 3′ of) the internal ribosomal entry site. The IRES thus provides for translation independent of a 5′ cap structure, and independent of the 5′ end of the mRNA. An IRES sequence of the vectors, thus, contain at least a minimal cis-acting sequences required for initiation of translation of an operably linked coding region.

Any of a variety of naturally-occurring or synthetic (e.g. recombinant) IRES sequences can be used in the vectors described herein. Naturally occurring IRES sequences are known in the art and include, but are not limited to, IRES sequences derived from rhinovirus, apthovirus, cardiovirus, encephalomyocarditis, enterovirus, adenovirus, influenza virus, herpes virus, cytomegalovirus, HIV virus, mengovirus, bovine viral diarrhea virus (BVDV), hepatitis A virus, hepatitis B virus, hepatitis C virus, GTX, Cyr61a, Cyr61b, poliovirus, the immunoglobulin heavy-chain-binding protein (BiP), immunoglobulin heavy chain, a picornavirus, murine encephalomyocarditis virus, poliovirus, and foot and mouth disease virus, and the like. Other IRES sequences useful in the vectors include those reported in WO 96/01324; WO 98/49334; WO 00/44896; and U.S. Pat. No. 6,171,821, each of which references is incorporated herein by in its entirety.

Fragments, mutants, variants and derivatives of naturally occurring IRES sequences may be employed in the present invention provided they retain the ability to initiate translation of an operably linked coding sequence located 3′ of the IRES.

Non-IRES Translation Initiation Signals

Translation initiation signals other than IRESs can be used in the expression bicistronic expression cassettes and vectors described herein. An example of a non-IRES translation initiation signal is a polyribosomal slippage sequence. Examples of polynucleotides encoding a non-IRES translation initiation signal include, but are not necessarily limited to the 2A region of picrornaviruses (see, e.g., de Felipe et al. (2003) J Gen Virol 84, 1281-1285), such as the 2A region of aphthovirus foot-and-mouth disease virus (FMDV) (see, e.g., Donnelly et al. Journal of General Virology (2001), 82, 1013-1025; Furler et al. (2001) Gene Ther 8, 864-873; Zhang et al. (2004) Sci China C Life Sci 47, 74-81), encephalomyocarditis virus (ECMV) (see, e.g., De Felipe et al. (2000) Hum Gene Ther 11, 1921-1931), aphthovirus, cardiovirus (Ryan et al. Bioorganic Chemistry 27, 55-79 (1999), simian picornaviruses (e.g., simian virus (SV) 2, SV16, SV18, SV42, SV44, SV45, and SV49) (see, e.g., Oberste et al. (2003) Virology 314, 283-293; the 2A region of lentiviruses (see, e.g., Chinnasamy et al. (2006). J. Virol 3, 14); and the like.

Promoters and Other Vector Elements

Any suitable promoter can be used to provide for expression of the coding sequences of the bicistronic vectors described herein. By “promoter” is meant at least a minimal sequence sufficient to direct transcription. “Promoter” is also meant to encompass those promoter elements sufficient for promoter-dependent gene expression controllable for cell-type specific, tissue-specific or inducible by external signals or agents; such elements may be located in the 5′ or 3′ regions of the native gene. Promoter(s) used in the constructs can be endogenous to the gene for which they drive expression, or may be heterologous In general, the promoter is operably linked to the 5′ most cistron, often referred to herein as the first cistron, of the bicistronic vector. In an embodiment of particular interest, the first cistron encodes a VEGF molecule. The promoter is usually a promoter heterologous to the VEGF coding sequence present in the vector, and can be a constitutive or inducible promoter. Of particular interest are strong promoters, such as those derived from a viral promoter that functions in eukaryotic cells. Exemplary promoters include a promoter from cytomegalovirus (CMV), mouse mammary tumor virus (MMTV), Rous sarcoma virus (RSV), or adenovirus. Further exemplary promoters include the promoter from the immediate early gene of human CMV (Boshart et al., Cell 41:521-530, 1985) and the promoter from the long terminal repeat (LTR) of RSV (Gorman et al., Proc. Natl. Acad. Sci. USA 79:6777-6781, 1982). The promoter can also be provided by, for example, a 5′UTR of a retrovirus. As discussed above, in dual expression cassette constructs, the two promoters can be selected to be the same or different with respect to transcriptional activity.

Alternatively, the promoter used may be a strong general eukaryotic promoter such as the actin gene promoter. Alternatively, the promoter may be a tissue-specific promoter (e.g., muscle-specific promoter, cardiac tissue-specific promoter, endothelial cell-specific, and the like).

In another embodiment, the promoter is a regulated promoter, such as a tetracycline-regulated promoter, expression from which can be regulated by exposure to an exogenous substance (e.g., tetracycline.).

Other components which can be present in the vectors described herein include, for example, a packaging signal (e.g., to facilitate encapsidation by a virus), selectable or detectable marker (e.g., an antibiotic resistance gene (such as an ampicillin resistance gene) or β-galactosidase) aid in selection or identification of cells containing and/or expressing the construct, an origin of replication for stable replication of the construct in a bacterial cell (preferably, a high copy number origin of replication), a nuclear localization signal, or other elements which facilitate production of the DNA construct, the protein(s) encoded thereby, or both.

Methods of Use

The vectors described herein can be delivered using non-viral or viral methods, and may be used in in vivo or ex vivo applications.

Viral-Based Delivery Modalities for Expression Constructs

For delivery using viral vectors, any of a number of viral vectors can be used. For example, the vectors can be based on retroviral vectors; adenoviral-vectors; adeno-associated viral vectors; lentiviral vectors; sindbis vectors; and the like. Promoters that are suitable for use with these vectors include the retroviral LTRs (e.g., Moloney retroviral LTR), CMV promoter, HSV promoter, and the like. Viruses suitable for delivery of the vectors described herein are generally replication incompetent or otherwise attenuated. Methods for production of suitable viral vectors are well known in the art.

Replication incompetent free virus can be produced and injected directly into the subject to provide for recombinant modification of cells in situ, or can be used in transduction of a cell (e.g., autologous or allogeneic) ex vivo which is then administered to the subject.

Non-Viral Delivery Modalities for Expression Constructs

For non-viral delivery, the vector can be formulated to facilitate delivery into a target cell. Constructs in the context of such non-viral delivery is often referred to as “naked DNA”. By “naked DNA” or “naked nucleic acid” is meant a nucleic acid molecule that is not contained within a viral particle. While not necessary in all applications, naked nucleic acid can optionally be associated (e.g. formulated) with means for facilitating delivery of the nucleic acid to the site of the target cell (e.g., means that facilitate travel into the cell, protect the nucleic acid from nuclease degradation, and the like) and/or to the surface of the target epithelial cell (e.g., adhesive microparticles, ligand-delivery complexes, and the like)

As noted above, the vector can be associated with agents to promote delivery and/or uptake of the vector into a target cell. For example, the vector can be incubated with a synthetic gene transfer molecule such as a polymeric DNA-binding cation such as polylysine, protamine, and albumin; linked to cell targeting ligands; or otherwise modified to facilitate delivery and/or uptake by the target cells. Further non-viral delivery suitable for use includes mechanical delivery systems such as the biolistic approach, as described in Woffendin et al., Proc. Natl. Acad. Sci. USA (11994) 91(24): 11581-11585. Moreover, the coding sequence and the product of expression of such can be delivered through deposition of photopolymerized hydrogel materials. Other conventional methods for gene delivery that can be used for delivery of the coding sequence include, for example, use of hand held gene transfer particle gun, as described in U.S. Pat. No. 5,149,655; use of ionizing radiation for activating transferred gene, as described in U.S. Pat. No. 5,206,152 and PCT application WO 92/11033.

In one embodiment, the vectors described herein can be provided in a liposome formulation. Liposomal preparations include cationic (positively charged), anionic (negatively charged) and neutral preparations, with cationic liposomes particularly preferred. Exemplary cationic liposomes include N[1-2,3-dioleyloxy)propyl]-N,N,N-triethylammonium (DOTMA) liposomes are available under the trademark Lipofectin, from GIBCO BRL, Grand Island, N.Y. (See, also, Felgner et al. Proc. Natl. Acad. Sci. USA (1987) 84:7413-7416). Other commercially available liposomes include transfectace (DDAB/DOPE) and DOTAP/DOPE (Boerhinger). Other cationic liposomes can be prepared from readily available materials using techniques well known in the art. See, e.g., Szoka et al. Proc. Natl. Acad. Sci. USA (1978) 75:4194-4198; PCT Publication No. WO 90/11092 for a description of the synthesis of DOTAP (1,2-bis(oleoyloxy)-3-(trimethylamm-onio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See, e.g., Straubinger et al. in Methods of immunology (1983), Vol. 101, pp. 512-527; Szoka et al. Proc. Natl. Acad. Sci. USA (1978) 75:4194-4198; Papahadjopoulos et al. Biochim. Biophys. Acta (1975) 394:483; Wilson et al. Cell (1979) 17:77); Deamer and Bangham Biochim. Biophys. Acta (1976) 443:629; Ostro et al. Biochem. Biophys. Res. Commun. (1977) 76:836; Fraley et al. Proc. Natl. Acad. Sci. USA (1979) 76:3348); Enoch and Strittmatter Proc. Natl. Acad. Sci. USA (1979) 76:145); Fraley et al. J. Biol. Chem. (1980) 255:10431; Szoka and Papahadjopoulos Proc. Natl. Acad. Sci. USA (1978) 75:145; and Schaefer-Ridder et al. Science (1982) 215:166. Other exemplary liposome compositions include those described in U.S. Pat. No. 5,422,120, WO 95/13796, WO 94/23697, WO 91/14445 and EP 524,968 B1.

The vectors described herein (regardless of formulation, or whether provided in viral or non-viral delivery modalities) are generally administered in a pharmaceutical composition which will generally contain sufficient nucleic acid material to produce a therapeutically effective amount of the analog or analogs, as described above. For purposes of the present invention, an effective dose will be from about 0.05 mg/kg to about 50 mg/kg of the DNA constructs in the individual to which it is administered.

Cell-Based Modalities for Delivery

Delivery of VEGF and PDGF can be accomplished by administering a recombinant cell containing an expression cassette (e.g., in the form of a biscistronic vector or dual expression cassette vector) to a site in a subject. Such cell-based modalities include those suitable for ex vivo therapy. By “ex vivo” it is meant that cells (e.g., isolated cells or cells in tissue) are modified in a culture (e.g., outside of a subject's body), and the genetically modifiedl cells are introduced to a subject. The expression cassette can be introduced into the cell under cell culture conditions, e.g., using standard techniques. Examples of techniques include but are not necessarily limited to: viral infection, transfection, conjugation, protoplast fusion, electroporation, particle gun technology (e.g., where the construct is administered in vivo), calcium phosphate precipitation, direct microinjection, viral vector delivery, ultrasound (e.g., where the construct is administered in vivo), compaction, and the like. Of interest are genetic modification techniques that do not require cell division as a condition for introduction of a recombinant polynucleotide.

Recombinant cells can be provided in a pharmaceutically acceptable formulation and provided in a sterile vial. Recombinant cells can be prepared and stored (e.g., in liquid nitrogen) until time of use. The cells used for production of recombinant cells may be any suitable cell that is compatible for subsequent implantation, e.g., compatible for implantation at a site in a subject (e.g., a mammal, particularly a human) at which increased anigiogenesis and/or vasculogenesis is desired (e.g., a site at which vessel production is desired). The cell is generally selected according to a variety of factors such as compatibility with the expression cassette to be used (e.g., a cell in which the promoter(s), translation initiation signal, and other cassette elements are translated and/or transcribed at a desired level), the subject to receive the implant, and the desired effect (e.g., an expression level that provides for a desired level of angiogenesis and/or vasculogenesis).

The cell can be a primary cell or from a suitable cell line. As used herein, the term “primary cell” generally refers to cells present in a suspension of cells isolated from a vertebrate tissue source (e.g., prior to being plated onto a surface of a tissue culture dish), cells present in an explant derived from tissue, and cultures (including suspensions) of such cells that are not immortalized. Primary cells and progeny thereof can be obtained from the subject to be treated (for autologous therapy) or from a donor subject (e.g., a non-recipient subject of the same species). Cells for genetic modification, which can then be administered to the host as in ex vivo therapy, can be obtained from a variety of tissues and include all cell types, with those that can be maintained in culture being of interest. For example, the cells used for production of recombinant cells co-expressing VEGF and PDGF as described herein may be derived from a tissue origin that is the same or similar to that to be modified (e.g., muscle, cardiac tissue, etc.), and further may be undifferentiated (e.g., pluripotent or multipotent cells, which are replicated in their undifferentiated state or cultured to provide for production of differentiated cells) or terminally differentiated (e.g., assuming a 45 day cell lifespan).

Exemplary host cells for use in the methods of the invention include muscle cells (e.g., myoblasts, mature skeletal muscle cells, endothelial cells, and the like). Further exemplary cells for use as host cells in the methods described herein include embryonic stem cells (e.g., human embryonic stem cells), as well as multipotent progenitor cells or adult stem cells or other cells suitable for use in a cell-based therapy (e.g., to promote tissue regeneration or engraftment at a desired site. Examples include mesenchymal stem cells. Examples include progenitor cells or adult stem cells of muscle (e.g., skeletal muscle, smooth muscle, and the like), bone (e.g., osteoblasts and bone-marrow-derived mesenchymal stem cells), hepatocytes, endothelial cells, and the like. Adult stem cells and progenitors of cardiac cells (e.g., cardioprogenitor cells), hematopoietic cells (e.g., hematopoietic progenitors and adult stem cells of bone marrow), and adipose-derived mesenchymal stem cells being of interest other).

The recombinant cells can be provided a population of cells, which population of cells can be heterogenous, e.g., with respect to whether the cell is recombinant for a PDGF-VEGF expression cassette as described herein, with respect to cell type or tissue origin; with respect to differentiation state (e.g., the population may be a mixture of undifferentiated cells (e.g., embryonic stem cells, adult stem cells, progenitor cells; or more generally pluripotent cells (e.g., embryonic stem cells) and/or multipotent cells); with respect to expression as a result of transient expression of a construct or expression as a result of genomic integration of the construct; and the like.

For example, a population of cells comprising a recombinant cell genetically modified to express a VEGF-PDGF expression cassette described herein can be generated by introducing a polynucleotide encoding a VEGF-PDGF expression cassette into a cell or population of cells (e.g., a cell or population of cells obtained from a source not limited to a subject to be treated or a donor subject) by any suitable means (e.g., transfection, electroporation, etc.). As a result of, for example, efficiency of the method used to genetically modify the population of cells and other factors (e.g., transfection efficiency), the resulting cells (i.e., parent cells and/or their progeny) can constitute a population of cells in which genetically modified cells containing the expression cassette represent at least 5%, at least 10%, at least 20%, at least 25%, at least 40%, at least 50%, at least 55%, at least 60%, at least 65%, at least 70% at least 75%, at least 80%, at least 90%, or at least 95% of the total cells in the population. In some examples, the resulting cell population contains from about 5% to 100%, from about 10% to 95%, from about 20% to 90%, from about 25% to 85%, from about 30% to 80%, or from about 40% to 75% recombinant cells. Non-recombinant cells in the population can represent cells (i.e., parent cells and/or their progeny) in which genetic modification was not achieved (e.g., untransfected cells), non-viable cells, and the like.

Optionally, genetically modified cells can be separated from non-genetically modified cells and/or non-viable cells (e.g., by selection, sorting, or other means) so as to provide a cell population that is enriched for the genetically modified cells. Exemplary cell populations include those that are enriched with 60%, 65%, 70%, 75%, 80%, 85%, 90%, 95% or more of cells in the population being genetically modified cells. Thus the disclosure herein contemplates, for example, a cell population that provides a cell-based therapy generating by genetically modifying a target stem cell (adult or embryonic) or progenitor cell to achieve an acceptable transfection efficiency, and generating a cell population that is, for example, about 60%, 70%, 80%, or 90% or more pure for the genetically modified cells. Such cells can be provided in a manner suitable for administration, e.g., in a pharmaceutically acceptable carrier, in a sterile vial, with a device for introduction into a subject (e.g., in a syringe), and the like.

Recombinant cells can be provided, ready for administration in a syringe or other device for injection. Guidance as to dose and dosage can be obtained from the knowledge of the ordinarily skilled artisan, and may involve, for example, administration of from about 1×10⁶ to 100×10⁶ cells in a volume of about 10 μl to about 1000 μl. Administration may be accomplished by a single or multiple injections. For example, cells may be delivered to the subject at a desired site(s) with from about 10 to 100 injections per local treatment site being contemplated. Methods for introduction of cells at a desired site are well known.

Devices

Delivery of VEGF and PDGF can be accomplished by coating a device, such as a stent, with composition described herein (e.g., an expression cassette-containing construct, and/or with recombinant cells containing such a construct. The compositions for delivery of VEGF and PDGF described herein can be provided, for example, in connection with a vascular prosthesis (e.g., synthetic vessel or graft), a stent (e.g., coated on a surface of a stent), catheter (e.g., balloon catheter), a bandage (e.g., provided on at least a wound-contacting region of a bandage, a suture (e.g., provided on at least a wound-contacting area of a suture), an implantable device, a gel (e.g., to provide for sustained release at a site of administration, which site can be topical or internal (e.g., at an internal wound, as in a surgical wound) and the like. The devices can be composed of a biodegradable material (and thus remain at or near the site of administration until degraded) or may be removed.

In another example, VEGF-PDGF delivery compositions (vectors and/or recombinant cells) are provided in conjunction with a support, such as a scaffold, which may comprise a biologically compatible, biodegradable or non-degradable material (e.g., as used in tissue engineering of bone and tissues, e.g., soft). A variety of biologically compatible materials are known. Exemplary materials include, but are not limited to, hydrogels, collagen gels, poly(lactide) [PLA] and poly(lactide-co-glycolide) [PLGA] fiber matrices, polyglactin fibers, calcium alginate gels, polyglycolic acid (PGA meshes, and other polyesters such as poly-(L-lactic acid) [PLLA], and polyanhydrides. The materials used as scaffolds may be may be naturally-occurring (e.g., agarose, alginate) or may be synthetically prepared or modified. Crosslinks (e.g., covalent bonds) between adjacent macromolecules in the support (e.g., in a hydrogel) and provide for differing degrees of aqueous solubility or insolubility of these hydrogels.

The scaffolds may be composed of porous or nonporous materials. For example, and particularly where the scaffold is to be used in the context of a bone graft, the scaffold may be composed of anorganic bone mineral (ABM), natural or synthetic hydroxyapatites, calcium phosphates, and other inorganic or organic compositions. The scaffolds may be featured into desired shapes or blocks that may be machined to obtain specific shapes for particular applications.

The scaffolds containing VEGF-PDGF delivery compositions (vectors and recombinant cells) can be produced in, and maintained in, a culture until time of use. Further where the scaffolds include recombinant host cells for expression of a PDGF-VEGF expression construct as described herein, the cells can be any of a variety of host cells, with those useful in tissue engineering applications being of particular interest. For example, the recombinant host cell can be an embryonic stem cells, adult stem cells, progenitor cells, mature (adult) cells, with specific examples of such cells provided above. Exemplary cells include progenitor cells or adult stem cells of muscle (e.g., skeletal muscle, smooth muscle, and the like), bone (e.g., osteoblasts and bone-marrow-derived mesenchymal stem cells), hepatocytes, endothelial cells, and the like. Adult stem cells and progenitors of cardiac cells (e.g., cardioprogenitor cells), hematopoietic cells (e.g., hematopoietic progenitors and adult stem cells of bone marrow), and adipose-derived mesenchymal stem cells being of interest other).

The vectors or recombinant cells can be provided on a surface of the device which is to contact a local site at which induction of angiogenesis and/or vasculogenesis is desired, or, such as in the case of biodegradable materials, contained in and/or on the device.

Delivery Methods

Once formulated, the compositions of the invention can be administered directly to the subject or, alternatively delivered ex vivo, to a host cell, which is then administered to the subject.

Methods to accomplish in vivo delivery of a nucleic acid of interest for expression in a target cell are known in the art. For example, and as discussed above, in vivo methods of DNA delivery normally employ either a biological means of introducing the DNA into the target cells (e.g., a virus containing the DNA of interest) or a mechanical means to introduce the DNA into the target cells (e.g., direct injection of DNA into the cells, liposome fusion, or pneumatic injection using a gene gun).

The amount of DNA introduced will vary according to the subject, the condition to be treated, the route of administration, and the like, and can be readily determined through routine methods. Generally, the amounts of introduced DNA can be extrapolated from the amounts of DNA effective for delivery and expression of the desired gene in an animal model. For example, the amount of DNA for delivery in a human is roughly about 100 times the amount of DNA effective in a mouse or rat model. For example, where the constructs described herein are delivered using a viral-based modality, administration can involve delivery (e.g., by injection at a local site) of from about 10⁶ to 10¹² viral particles, from 10⁷ to 10¹¹ viral particles, from about 10⁷ to 10¹⁰ viral particles, or from about 10⁸ to 10⁹ viral particles per dose. Guidance for dose and dosages can also be found in the art. The methods can be accomplished by delivery of the DNA in viral or non-viral modalities as single or multiple administration (e.g., single or multiple injections) at and/or around a local site, as well as at a combination of different sites (e.g., around and/or along a site of an area (e.g., an area of damage) at which increased angiogenesis is desired).

In ex vivo methods, host cells for ex vivo methods are selected and administered so as to provide for production of VEGF and PDGF at a desired site following introduction to the host, while minimizing adverse side effects (e.g., immune response to the recombinant cells introduced). The host cell can be autologous to the subject, or may be allogeneic (e.g., obtained from a donor subject of the same species). The cells may be derived from a tissue origin that is the same or similar to that to be modified (e.g., muscle, cardiac tissue, etc.). Exemplary host cells for use in the methods of the invention include muscle cells (e.g., myoblasts, mature skeletal muscle cells, endothelial cells, and the like). Further exemplary cells include embryonic stem cells (e.g., human embryonic stem cells), as well as progenitor cells or adult stem cells (e.g., progenitor cells or adult stem cells of muscle (e.g., skeletal muscle, smooth muscle, and the like), bone (e.g., osteoblasts), hepatocytes, endothelial cells, and the like), and other cells suitable for use in a cell-based therapy (e.g., to promote tissue regeneration or engraftment at a desired site).

Methods for the ex vivo delivery and introduction of genetically modified cells into a subject are known in the art. Generally, methods to accomplish introduction of a vector into a cell ex vivo will include dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art. Methods for introduction or implantation of cells modified as described herein are well known in the art.

The polynucleotide-based modified cell can be introduced by various routes of administration and at various sites (e.g., muscle (e.g., smooth muscle, skeletal muscle), cardiac tissue, skin, eye (e.g., cornea), and the like. The genetically modified cells produce VEGF and PDGF while implanted, and facilitate angiogenesis and/or vasculogenesis.

The number of recombinant cells introduced will vary according to the subject, the condition to be treated, the route of administration, and the like, and can readily be determined through routine methods.

Routes of Administration

The compositions of the invention (e.g., vectors, virally-encapsulated vectors, and/or recombinant cells containing vectors described herein) can be administered by any suitable route compatible with the form in which the vector is to be delivered, the site to be treated, and the like so as to provide for a desired ratio of PDGF:VEGF within a microenvironment at and surrounding a treatment site. For example, the compositions can be administered to a local site. Such local sites can any site at which induction of angiogenesis may be desired as a therapeutic intervention, including preventative or palliative intervention. Local sites can be topical (e.g., as in a topical wound) or internal (e.g., deep tissue (e.g., as in a surgical wound), associated with an organ or limb having or susceptible to damage due to hypoxia and/or ischemia (e.g., cardiac tissue, muscle, and the like), and the like).

As described above, administration of the VEGF-PDGF compositions described herein can also be accomplished by, for example, use of an implantable device, which may optionally be composed of biodegradable material (and thus remain at the site until dissolved) or may be removed. Such devices can include, for example, a vascular prosthesis (e.g., synthetic vessel or graft), stent, catheter (e.g., balloon catheter), scaffolds (as exemplified above), and the like.

The VEGF-PDGF compositions can be provided on a surface of the device or, such as in the case of biodegradable materials, contained in and/or on the device (e.g., as in a gel, such as a sustained release formulation).

Conditions Amenable to Treatment

The methods and compositions described herein can be used to treat a variety of conditions that would benefit from stimulation of angiogenesis, stimulation of vasculogenesis, increased blood flow, and/or increased vascularity. Of particular interest is the stimulation of angiogenesis in vivo to effect increase in blood flow, increased capillary density, and/or increased vascularity within, adjacent, or around an ischemic site, with no or little aberrant angiogenesis. Accordingly, the methods and compositions described herein can be administered to any subject (e.g., human) in need of therapy and having a condition amenable to treatment by delivery of VEGF and PDGF (e.g., to induce angiogenesis and/or vasculogenesis).

Examples of conditions and diseases amenable to treatment according to the methods of the invention include any condition associated with an obstruction of a blood vessel, e.g., obstruction of an artery, vein, or of a capillary system. Specific examples of such conditions or diseases amenable to treatment include, but are not necessarily limited to, stroke, wounds (e.g., head injury), coronary occlusive disease, carotid occlusive disease, arterial occlusive disease, peripheral arterial disease, atherosclerosis, myointimal hyperplasia (e.g., due to vascular surgery or balloon angioplasty or vascular stenting), thromboangiitis obliterans, thrombotic disorders, vasculitis, and the like. Examples of conditions or diseases that can be prevented using the compositions described herein include, but are not necessarily limited to, heart attack (myocardial infarction) or other vascular death, death or loss of limbs associated with decreased blood flow, and the like. Further conditions include treatment of ischemic limb disease (such as common in diabetics) and regeneration of damaged cardiac tissue.

Other forms of therapeutic angiogenesis contemplated herein include, but are not necessarily limited to, the use of the compositions described herein to accelerate healing of wounds or ulcers; to improve the vascularization of skin grafts or reattached limbs (e.g., so as to preserve function and viability); to improve the healing of surgical anastomoses (e.g., as in re-connecting portions of the bowel after gastrointestinal surgery); provide for liver regeneration; provide for treatment of hypertension (e.g., portal hypertension); and other applications which will be readily appreciated by the ordinarily skilled artisan upon reading the present disclosure. In one example, the VEGF-PDGF compositions (vectors and recombinant cells) can be used in tissue engineering applications, e.g., by introduction of a biologically compatible scaffold at a desired site in a subject, where the scaffold contains recombinant host cells (e.g., embryonic stem cells, adult stem cells, progenitor cells, mature (adult) cells, etc.) that contain a VEGF-PDGF construct as described herein.

Kits

Kits with unit doses of the vectors for delivery of VEGF and PDGF (bicistronic expression cassette vectors, and dual expression cassette vectors), which are optionally provided in connection with a delivery modality (e.g., virally-encapsidated, in a liposome formulation, in a genetically modified cell, in conjunction with a scaffold or other device), and which are usually in formulations suitable for administration by a desired route to a subject (particularly a human), are provided. In such kits, in addition to the containers containing the unit doses will be an informational package insert describing the use and attendant benefits of the compositions in treating a condition of interest.

### EXAMPLES

The following examples are put forth so as to further provide those of ordinary skill in the art a disclosure and description of how to make and use various aspects of the present invention, and are not intended to limit the scope of what is regarded as the invention, nor are they intended to represent that the examples below are all or the only actual reduction to practice.

Methods and Materials

The following methods and materials were used in the Examples set out below.

Cell Culture: Primary myoblasts isolated from C57BL/6 mice and transduced to express the β-galactosidase marker gene (lacZ) from a retroviral promoter (Rando et al. J Cell Biol 125, 1275-87 (1994)) were further infected at high efficiency with four successive rounds of retroviruses carrying the cDNA for murine VEGF₁₆₄ (Springer, M. L., et al. 1988. Mol Cell 2, 549-58; Springer, M. L. et al. 1997. Somat Cell Mol Genet 23, 203-9) or human PDGFb (hPDGFb) or both. The hPDGFb construct was composed of an optimal Kozack sequence (ACC) positioned immediately 5′ of the wild-type nucleotide sequence of the hPDGF described at GenBank Acc. No. NM_{—}002608.1. The isolation and characterization of early passage myoblast clones homogeneously expressing precise VEGF levels have been previously described (Ozawa, C. R. et al. 2004. J Clin Invest 113, 516-27). All myoblast populations were cultured in 5% CO₂ on collagen-coated dishes as previously described (Rando, T. A. et al. 1994. J Cell Biol 125, 1275-87) and (Springer, M. L. et al. 1997. Somat Cell Mol Genet 23, 203-9).

VEGF₁₆₄ and PDGF-BB ELISA measurements: Cell culture supernatants were quantified for VEGF₁₆₄ and PDGF-BB protein using an ELISA kit (R&D Systems, Minneapolis, Minn.): 1 ml of medium was harvested from myoblasts in one 60 mm dish, following a four-hour incubation, filtered and analyzed in duplicate. Medium was supplemented with 10 μg/ml heparin to prevent retention of PDGF-BB on the cell surface. Results were normalized for the number of cells and time of exposure to medium. Four dishes of cells were assayed per cell type (n=4).

Implantation of myoblasts into mice: 6-8 week-old, male SCID CB. 17 mice (Taconic, Germantown, N.Y.) were treated in accordance with institutional guidelines, after approval from Stanford University ethical committee. SCID mice were used to avoid an immunologic response to myoblasts expressing xenogenic proteins. Myoblasts were dissociated in trypsin and resuspended in PBS with 0.5% BSA. 5×10⁵ myoblasts in 5 μl were implanted into the tibialis anterior muscle in the calf, or into the posterior auricular muscle, midway up the dorsal aspect of the external ear, using a syringe with a 29.5-gauge needle.

Tissue staining: The entire vascular network of the ear could be visualized following intravascular staining with a biotinylated Lycopersicon esculentum lectin that binds the luminal surface of all blood vessels, as previously described (Ozawa et al. J Clin Invest 113, 516-27 (2004); Thurston et al. Nat Med 6, 460-3. (2000); Thurston et al. Science 286, 2511-4. (1999)). Mice were anesthetized, lectin was injected intravenously and 2 minutes later the tissues were fixed by vascular perfusion of 1% paraformaldehyde and 0.5% glutaraldehyde in PBS pH 7.4. Ears were then removed, bisected in the plane of the cartilage, and stained with X-gal staining buffer (1 mg/ml 5-bromo-4-chloro-3-indoyl-β-D-galactoside, 5 mM potassium ferricyanide, 5 mM potassium ferrocyanide, 0.02% Nonidet P-40, 0.01% sodium deoxycholate, 1 mM MgCl₂, PBS pH 7.4). Tissues were stained using avidin-biotin complex-diaminobenzidine histochemistry (Vector Laboratories, Burlingame, Calif.), dehydrated through an alcohol series, cleared with toluene and whole-mounted on glass slides with Permount embedding medium (Fisher Scientific, Fair Lawn, N.J.). Vascular morphology was analyzed at 4 days, 1, 2 and 4 weeks for specific experiments.

For tissue sections, mice were anesthetized and sacrificed by cervical dislocation. Tibialis anterior muscles and whole ears were harvested, embedded in OCT compound (Sakura Finetek, Torrance, Calif.), frozen in freezing isopentane and cryosectioned. Tissue sections were then stained with X-gal (20 μm sections) or with H&E (10 μm sections) as described previously (Rando, T. A. et al. 1994. J Cell Biol 125, 1275-87) and (Springer, M. L., et al. 1997. Gene delivery to muscle, John Wiley Sons, New York).

Immunofluorescence was performed essentially as previously described (Springer, M. L., et al. 1998. Mol Cell 2, 549-58). The following primary antibodies and dilutions were used: rat anti-mouse Platelet-Endothelial Cell Adhesion Molecule-1 (PECAM-1; Pharmingen, San Diego, Calif.) at 1:100; mouse anti-mouse alpha-Smooth Muscle Actin (α-SMA; ICN Biomedicals, Aurora, Ohio) at 1:400; rabbit anti-NG2 (Chemicon International, Temecula, Calif.) at 1:200; goat anti-collagen type IV (Chemicon International, Temecula, Calif.) at 1:200. Fluorescently labeled secondary antibodies (Molecular Probes, Eugene, Oreg.) were used at 1:100.

Vessel measurements: Vessel diameters and length densities were measured in whole mounts of ears stained with L. esculentum lectin as previously described (Thurston et al. 2000 Nat Med 6, 460-3). Briefly, vessel diameters were measured by overlaying a captured microscopic image with a square grid. Squares were chosen at random, and the diameter of each vessel (if any) in the center of selected squares was measured. Two to three hundred total vessel diameter measurements were obtained from 4-5 ears per group (n=4-5). Vessel length density was measured on 3-6 fields per ear and 4-5 ears per group (n=4-5) by tracing the total length of vessels in the fields and dividing it by the area of the fields. All image measurements were performed with AnalySIS D software (Soft Imaging System Gmbh, Münster, Germany).

Plasma leakage measurements: Evans blue assays were performed as previously described (Thurston. et al. 2000. Nat Med 6, 460-3; Thurston, G. et al. 1999. Science 286, 2511-4). Briefly, Evans blue dye (30 mg/kg in 100 μl PBS, J. T. Baker, Phillipsburg, N.J.) was injected iv. Four hours later, mice were perfused with 1% paraformaldehyde in 0.05 M citric acid, pH 3.5. Six-millimeter biopsy punches of ears were obtained (Sklar Instruments, West Chester, Pa.). Evans blue was extracted from tissue with formamide at 55° C. overnight and measured with a spectrophotometer at 610 nm. Plasma leakage was measured 4 days, 1 and 2 weeks after myoblast implantation and expressed as ng dye/mg tissue wet weight (n=5). Total leakage was also normalized to the total vascular surface induced by the different myoblast populations in similarly injected ears (n=4-5 ears per condition). Total vascular surface was estimated as the product of average vessel perimeter (π×average diameter, measured as described above) and total vessel length (vessel length density, measured as described above, multiplied by the total area of effect measured on low-magnification microscopic pictures). Normalized leakage was expressed as ng dye/mm² of vascular surface in the area of effect (n=5).

Hindlimb ischemia: Male C.B-17-SCID mice (16-20 weeks old) were obtained from the Stanford University Department of Comparative Medicine and treated according to the guidelines of the Stanford Administrative Panel on Laboratory Animal Care. Anesthesia was induced and maintained by isoflurane inhalation. Unilateral hindlimb ischemia was surgically induced by ligation and transection of the medial portion of the right superficial femoral artery distal to the deep femoral artery origin. Muscle blood velocimetry was assessed over 2 minutes before and after surgery using a calibrated laser Doppler-probe (Perimed-PF3/Perisoft-software, Perimed, Jarfalla, Sweden) positioned on the distal adductor muscle using a 3-dimensional micromanipulator stage as described (Jacobi et al. 2005 Circulation 111, 1431-8). In preliminary studies, laser Doppler velocimetry manifested greater variability than microsphere measurements, but reliably identified severe hypoperfusion. Specifically, postoperative blood flow values that were generally less than 40% of the contralateral leg correlated well when determined by laser Doppler or measured by microspheres in all 20 animals studied with both techniques. Accordingly, laser Doppler was used solely to document postoperative ischemia after surgery. Mice were randomized after surgery to receive either vehicle only or 8×10⁶ myoblasts suspended in 0.5% BSA/PBS at a concentration of 10⁸ cells/ml. 8 injections of 10⁶ cells each were performed into the distal thigh muscles.

Fourteen days after surgery, blood flow was measured using fluorescent microspheres as previously described (Jacobi. et al. 2005 Circulation 111, 1431-8) in 5-7 mice/group. Following median thoracotomy, 2×10⁵ 15 μm-diameter red fluorescent microspheres (Molecular Probes, Eugene, Oreg.) were continuously injected over 60 seconds into the beating left ventricle. The heart was cannulated and perfused at 110 mmHg with Tris-HCl buffer containing Na+, Ca++, Mg++ and 0.1% adenosine (2 minutes), followed by 1.5% formaldehyde (2 minutes) (Scholz et al. 2002. J Mol Cell Cardiol 34, 775-87). The muscle group of the thigh (adductor and quadriceps femoris groups) was excised, cut in mid-thigh, weighed, embedded in OCT-compound and snap frozen. Kidneys were analyzed as reference organs to confirm equivalent bilateral microsphere distribution. Microspheres were individually counted by direct fluorescence microscopy on 100 μm cryosections from the entire samples. Microsphere counts, normalized for muscle weight, from the ischemic leg were normalized to the counts in the contralateral, non-ischemic leg. Collateral vessels ≧30 μm in diameter were identified on cross sections of the proximal adductor muscle by co-staining for PECAM and α-SMA and quantified as previously described (Jacobi et al. 2005 Circulation 111, 1431-8; Heeschen et al. 2001 Nat Med 7, 833-9). Damaged muscle was analyzed on H&E stained cryosections of the calf muscles, and defined as either inflammation (mononucleated cell infiltrates) or necrosis (“ghost fibers” lacking nuclei) (Paek et al. 2002 J Vasc Surg 36, 172-9). Areas were manually drawn on digital images (5×objective) and quantified using calibrated software.

Statistics: Data are presented as means ±standard error. The significance of differences was evaluated using analysis of variance followed by the Bonferroni test. P<0.05 was considered statistically significant.

### Example 1

### Co-Implantation of VEGF- and PDGF-BB-Expressing Myoblasts

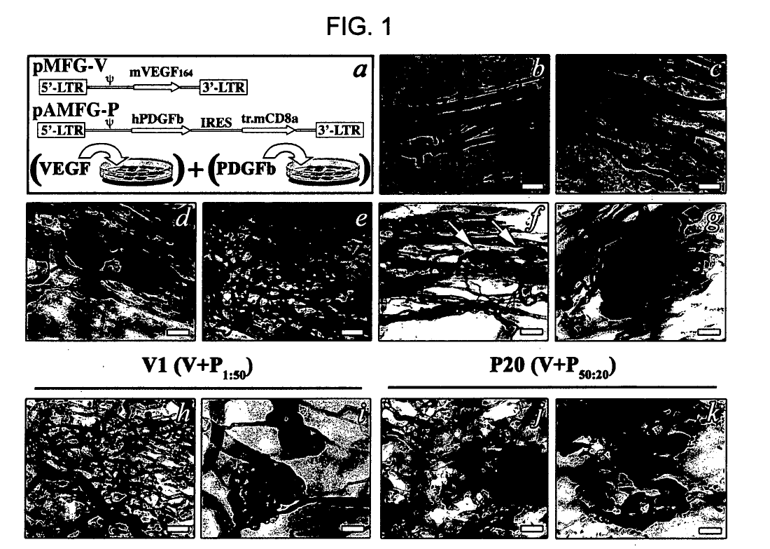

In order to study the effects PDGF-BB on VEGF-induced angiogenesis in vivo, the cDNA for these two factors were delivered to skeletal muscle by myoblast-mediated gene transfer. Significant advantages of this well-characterized system are sustained gene expression restricted to skeletal muscle, which is the target tissue of therapeutic angiogenesis approaches (Carmeliet et al. Nat Med 9, 653-60 (2003); Yla-Herttuala et al. Cardiovasc Med 14, 295-300 (2004)), and a precise control over the distribution of expression levels in vivo (Ozawa, C. R. et al. 2004. J Clin Invest 113, 516-27). Primary mouse myoblasts were retrovirally transduced to express LacZ and either murine VEGF₁₆₄ (Ozawa et al. 2004 J Clin Invest 113, 516-27; Springer et al. 1998 Mol Cell 2, 549-58) or human PDGFb (VZ and PZ cells, respectively, FIG. 1, Panel A). The hPDGFb sequence was linked to a truncated form of murine CD8a (trCD8a) as a cell-surface marker, in order to allow convenient FACS isolation of transduced cells. Control CD8Z cells expressed only trCD8a and LacZ. Transgene expression was confirmed by ELISA. VZ cells produced 112±10.4 ng/10⁶ cells/day of mVEGF₁₆₄, whereas PZ and control Z and CD8Z myoblasts secreted only background levels (0.3-0.5 ng/10⁶ cells/day). PZ cells produced 45.7±2.4 ng/10⁶ cells/day of hPDGF-BB.

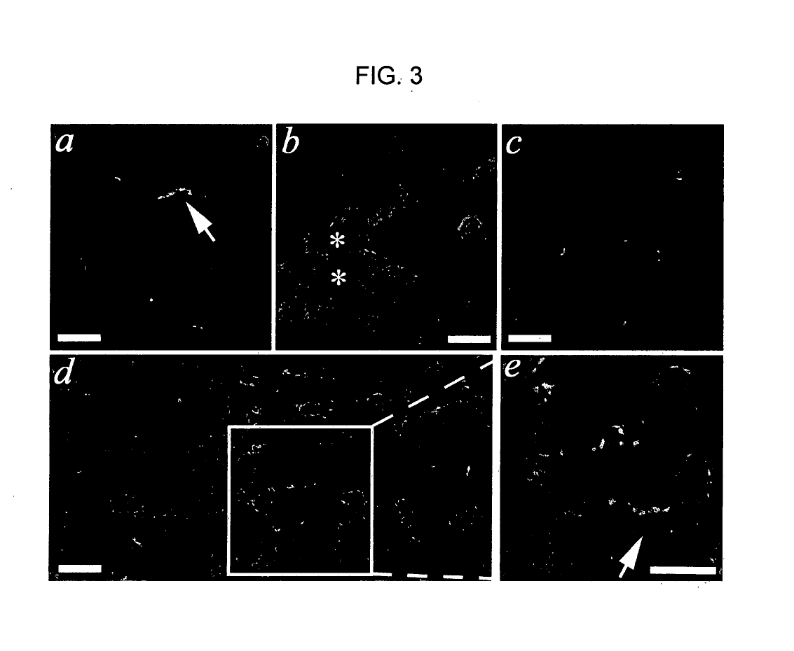

Engineered myoblast populations were implanted into the posterior auricular muscle and the 3-dimensional morphology of the induced vessels was analyzed after 2 and 4 weeks. Control CD8Z cells were shown to cause no perturbation of the vascular architecture at any time point (FIG. 1, Panel B), while VEGF-expressing cells induced the abundant growth of aberrant bulbous structures at 2 weeks, (FIG. 1, Panel D), demonstrating the evolution of hemangioma-like structures over time. PDGF-BB-expressing cells alone did not induce any new vessels, but the inter-capillary distance appeared to increase (FIG. 3, Panel C), as a result of the interstitial proliferation of NG2-positive, SMA-negative cells (FIG. 3, Panel D). Implanting the VZ and PZ populations together induced a new phenotype: the growth of highly-branched networks of short capillaries with homogeneous diameters (FIG. 1, Panel E). Although such capillary networks were widespread, bulbous angiomatous vessels were always found in some areas of the implantation sites (FIG. 1, Panels F-G), especially near the periphery, which were identical to those induced by high levels of VEGF alone.

It was hypothesized that areas of unopposed VEGF expression could lead to the persistence of aberrant angiogenesis and that an increase in the dose of delivered PDGF-BB relative to VEGF could prevent such occurrence. Therefore, the ratio of the two myoblast populations was varied in the implanted mixture. In one dose series, the amount of VZ cells was kept constant at 50% and the amount of PZ cells was decreased to 20%, 10%, 5% and 1% (P₂₀, P₁₀, P₅ and P₁, respectively), with the remainder being made up of LacZ-expressing control cells. In a complementary series, the PZ cells were maintained at 50% of the mixture while the amount of VZ cells was similarly varied down to 1% (V₂₀, V₁₀, V₅ and V₁ conditions). Only VEGF-induced aberrant structures could be found in the condition having the lowest amount of PZ cells (P₁), while in all other conditions, homogeneous-sized highly-branched capillary networks were induced (FIG. 1, Panels H and J). Bulbous pre-angiomatous bodies were also always present in all instances, even when only 1% of the cells expressed VEGF (FIG. 1, Panels I and K). The results demonstrate that the proportion of capillary networks induced by co-implantation of the two cell populations gradually increased with the percentage of PDGF-BB-expressing cells, from no PDGF-BB-expressing cells in the P₁ implantations (1% PZ+50% VZ) to the maximum number of PDGF-BB-expressing cells in P₅₀V₅₀ (50% PZ+50% VZ). Additionally, a gradual decrease was demonstrated in the conditions from V₂₀ to V₁ (data not shown) as the VEGF-expressing cells were reduced. However, no ratio of VEGF- to PDGF-BB-expressing cells could completely prevent the appearance of aberrant structures.

### Example 2

### Microenvironmental Co-Localization at Selected Ratio by Single-Vector Co-Expression

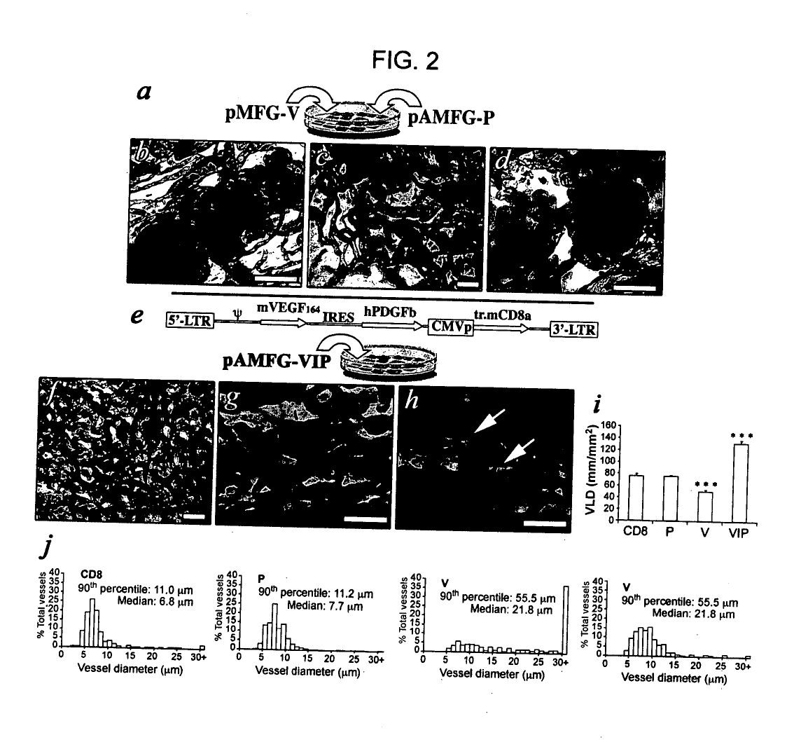

In order to achieve co-expression of VEGF and PDGF-BB in every transduced fiber, populations were generated in which every cell expressed both factors, either at random relative levels or in a selected, fixed ratio. In the first case (FIG. 2, Panel A), the VZ population was superinfected with the PDGF-BB retrovirus described above and the double-infected cells were sorted by FACS (VZ/P cells). Control VZ/CD8 cells were generated by superinfecting VZ cells with the empty CD8-expressing virus as above. Since the number of viral copies and integration sites are independent for the two constructs, co-expression is ensured in each cell, but at random ratios. Both populations produced similar average VEGF amounts as VZ cells (VZ/CD8=98.3±5.4 and VZ/P=132.7±1.7 ng/10⁶ cells/day) and VZ/P cells also secreted 54.6±3.4 ng/10⁶ cells/day of PDGF-BB, similarly to PZ cells.

The pAMFG-VIP construct (for Vegf-Ires-Pdgf, FIG. 2, Panel E) was generated in which the cDNAs of VEGF₁₆₄ and PDGFb are linked into a bicistronic cassette through the encephalomyocarditis virus Internal Ribosomal Entry Site (IRES), which ensures the translation of both sequences from a single messenger RNA. This allows the relative microenvironmental levels of VEGF and PDGF to be fixed regardless of the variability in viral infection efficiency and transcriptional activity of the random genomic integration sites. However, the expression of the two cistrons was not quantitatively equal because the second position was less expressed than the first. The amount of PDGF-BB produced by the VIP cells was 34±3% of that of VEGF on a molar basis. Two different populations were generated (VIP_{high} and VIP_{low}), expressing on average 58.9±5.8 and 107.9±6.2 ng VEGF/10⁶ cells/day respectively.

After implantation of VZ/P cells, the microenvironmental co-localization at random relative levels caused the formation of capillary networks (FIG. 2, Panel C). However, it could not completely prevent the appearance of some angiomatous vessels (FIG. 2, Panel D), which were the prevalent phenotype induced by VEGF alone from VZ/CD8 cells (FIG. 2, Panel B).

When the matching of microenvironmental relative levels was insured by implanting VIP populations, only highly-branched networks of homogeneous-size capillaries were induced in all sites (FIG. 2, Panels F-H). Both VIP populations yielded the same results, demonstrating that modulation of VEGF effects was independent of the VEGF dose. In particular, the VIP_{high} population expresses the same average VEGF levels as VZ and VZ/CD8 cells, which always induce hemangiomas. However, a single instance of aberrant bulbous vascular structures was not induced in 100% of the animals injected.

The data in the following examples refers to the use of VIP_{high} cells. VIP_{low} cell implantations yielded equivalent results.

### Example 3

### PDGF-BB Potentiates VEGF-Induced Angiogenesis

Vessel length density was quantified in the implantation sites as a measure of the total length of vessels in a given area independent of vessel diameter or number. PDGF-BB alone did not cause significant angiogenesis in implanted ear muscles when compared to control cells (75.7±1.0 vs 75.7±3.7 mm/mm², n.s.) as evident in FIG. 2, Panel I. VEGF alone was shown to cause a 33% reduction in vessel length density (50.7±2.6 mm/mm²), due to the replacement of normal capillaries with hyperfused bulbous structures, which provide only a short vascular length. However, the delivery of PDGF-BB and VEGF by expression from a contiguous polynucleotide (VIP) caused a 75% increase in vessel length density compared to controls and a 160% increase compared to VEGF (132.6±5.2 mm/mm², p<0.001 for all comparisons).

The analysis of vessel diameter distribution (FIG. 2, Panel J) demonstrates that normal capillaries in areas implanted with control cells (CD8) had a very homogeneous size with a median of 6.8 μm. PDGF-BB alone caused a modest enlargement of the pre-exisiting vessels, as the median diameter shifted to 7.7 μm. VEGF-induced vessels were highly heterogeneous, with a majority (67%) being dilated aberrant structures larger than 15 μm. Matched delivery of PDGF-BB and VEGF (VIP) prevented the appearance of glomeruloid bodies and only 5% of vessels were >15 μm, similar to the controls (CD8=5% and P=3%). These vessels are representative of normal arterioles and veins. The median vessel diameter was shown to further increase to 8.7 μm, while preserving the homogeneity of size distribution.

### Example 4

### PDGF-BB Delivery with VEGF by Expression from the Same Construct Promotes Vascular Maturation

The effects of stimulating pericyte recruitment were investigated by examining PDGF-BB and VEGF delivery (VIP) on the maturation of VEGF induced vessels in skeletal muscle two weeks after implantation with VZ, PZ, VIP_{high} or control CD8Z cells. Normal microvessels in control-implanted muscles were uniformly covered with pericytes, which stained positive for NG2 and negative for α-smooth muscle actin (SMA) and displayed the typical branched processes (arrow in FIG. 3, Panel A). The aberrant angiomatous vessels induced by uncontrolled VEGF expression were not devoid of mural cells, but lacked proper pericytes and acquired instead SMA+NG2-cells, which developed into a thick smooth muscle coat in the growing angioma sacks (FIG. 3, Panel B). PDGF-BB alone induced the proliferation of NG2+SMA-cells in the interstitium between muscle fibers, but no extra blood vessels (FIG. 3, Panel C). When high VEGF expression was linked with matched PDGF-BB levels (VIP_{high} cells), the induced vessels were uniformly covered by normal pericytes (FIG. 3, Panel D). Every CD31+ structure was invested by NG2+SMA-branched cells, which were embedded in the vascular basement membrane whose thin processes established tight cell-cell contact with the endothelium, as demonstrated in FIG. 3, Panel E by the co-localization of NG2 and laminin stains (arrow in FIG. 3, Panel E).

### Example 5

### PDGF-BB Co-Expression does not affect Vascular Induction by VEGF

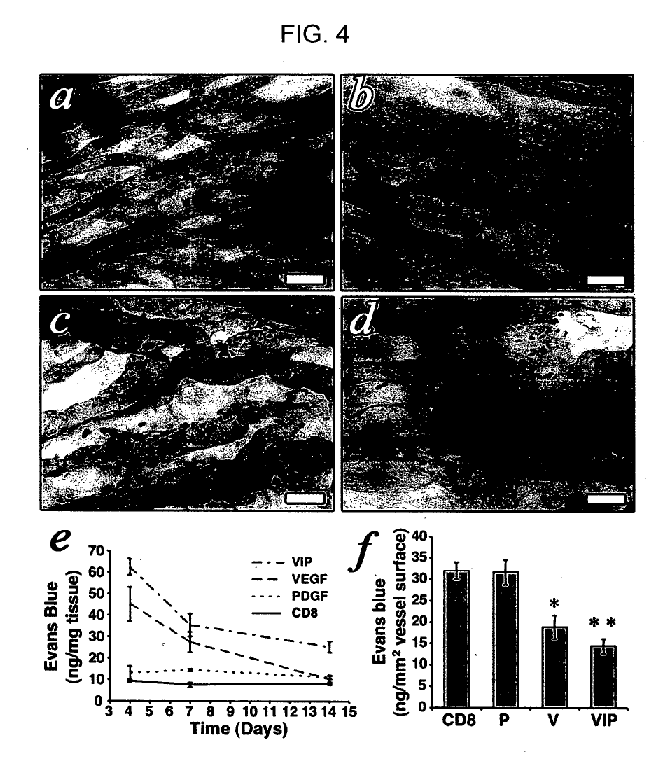

The effects of PDFG-BB on the early stages of VEGF-induced angiogenesis were investigated to better understand the mechanism of action of PDGF-BB on the early stages of VEGF-induced angiogenesis. The results demonstrate that PDGF-BB cells alone did not affect pre-existing vessels 4 days after implantation (FIG. 4, Panels A and B). In the initial stage of angiogenic induction, VEGF expression caused a marked enlargement of pre-existing vessels to form transient structures, similar to the previously described “mother vessels” (Pettersson, A. et al. 2000. Lab Invest 80, 99-115), which did not change in quality or quantity with matched-level PDGF-BB co-expression with VEGF from VIP_{high} myoblasts (FIG. 4, Panels C and D).

### Example 6

### PDGF-BB Delivery with VEGF by Expression from the Same Construct does not Exacerbate Vascular Leakage

PDGF-BB delivery with VEGF (VIP) did not inhibit the initial surge in VEGF-induced vascular leakage at 4 days and it did not increase plasma extravasation above basal levels by itself at any time-point (FIG. 4, Panel E) which is consistent with its lack of influence on the early angiogenic stages, as evident from above. VEGF-induced vascular leakage was transient and subsided by 2 weeks, synchronously with the acquisition of smooth-muscle coverage by the aberrant bulbous vessels despite their continuing evolution into angiomatous structures as previously described (Ozawa, C. R. et al. 2004. J Clin Invest 113, 516-27). VEGF-induced plasma extravasation decreased at 1 and 2 weeks in the presence of PDGF-BB but remained higher than VEGF alone.

PDGF-BB delivery with VEGF induced a 2.6-fold increase in vascular density compared to VEGF alone (FIG. 2, Panel I) over larger areas of effect. Therefore, the total vessel length in the implantation sites of VIP_{high} cells at 2 weeks was 14 times larger than with VEGF alone (874.4±236.6 mm vs 61.5±20.3 mm, p=0.027, n=4-5), whereas there was no significant difference in the initial response at 4 days. Since total plasma leakage depends directly on the surface of induced vasculature, the total amount of extravasated Evans blue (in ng) was normalized to the total vessel surface (in mm²) in the areas of effect 2 weeks after implantation of CD8, P, V or VIP_{high} myoblasts. The results (FIG. 4, Panel F) demonstrate that the plasma leakage of VIP_{high}-induced vessels was similar to that of VEGF-induced vessels after 2 weeks (14.4±1.7 and 18.8±2.7 ng/mm², p=n.s.), with both significantly less than leakage from vessels in areas implanted with CD8- and PDGF-expressing myoblasts (32.0±0.0 and 31.7±0.9 ng/mm², p<0.05).

### Example 7

### Blood Flow Recovery and Collateral Formation in Hindlimb Ischemia are Enhanced by Co-Delivery of PDGF and VEGF

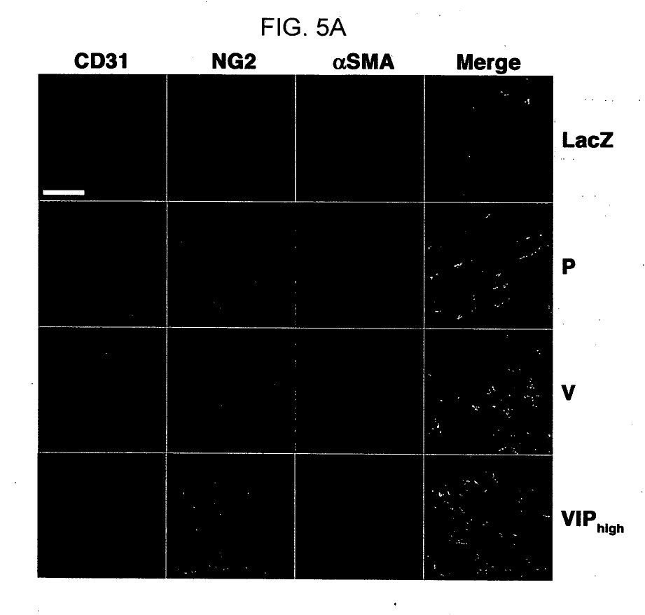

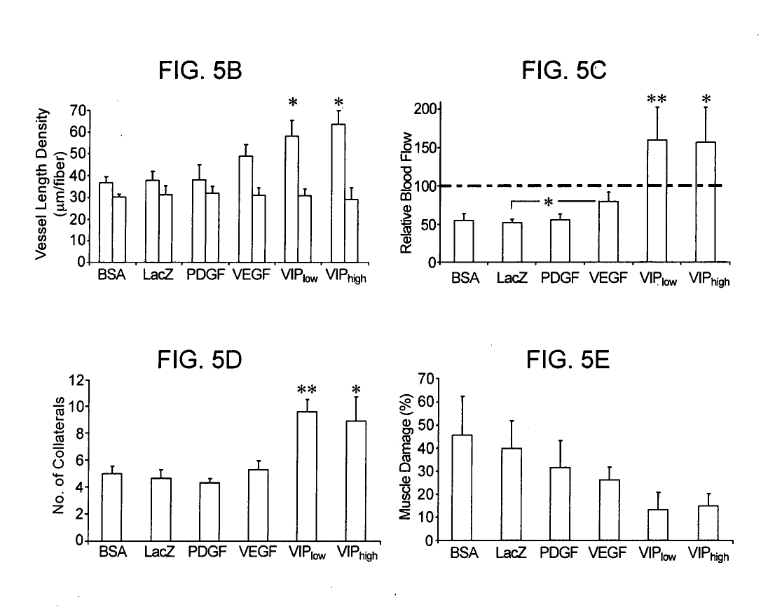

The effects of matched-level PDGF-BB co-delivery in a mouse model of hindlimb ischemia (Jacobi, J. et al. 2005. Circulation 111, 1431-8) was tested to determine the efficiency of newly-induced microvessels to increase regional blood flow. Ischemia caused a slight increase in vessel length density compared to the contralateral non-ischemic leg (BSA and LacZ controls; FIG. 5B). PDGF-BB delivery did not induce the growth of any further capillaries in ischemic muscle (FIGS. 5A-5B), thereby confirming the results obtained in non-ischemic tisssue. VEGF alone was found to increase vessel length density by about 30% over control cells (48.8±5.4 vs 37.7±4.2 μm/fiber) and PDGF-BB co-delivery by the two VIP populations further increased vascular growth to 58.1±7.2 and 63.4±6.4 μm/fiber (FIG. 5B, p<0.05).

Blood flow was measured by the fluorescent microsphere method and was expressed as a percentage of the contralateral non-ischemic leg of the same mouse. Two weeks after induction of ischemia, PDGF-BB alone did not improve blood flow compared to injection of BSA or control LacZ cells (56% vs 54.9% and 51.7% of non-ischemic flow respectively, FIG. 5C). VEGF alone induced a moderate increase in blood flow to 79.8% of non-ischemic levels, which was significant compared to control cells (p<0.05) and borderline significant versus PDGF-BB alone (p=0.067). However, matched-level co-delivery of both factors dramatically improved perfusion of the ischemic hindlimbs to more than non-ischemic levels (VIP_{low}=159.6% and VIP_{high}=156.8%, p<0.05 for all comparisons in FIG. 5C). This flow increase was greater than the flow increase induced by optimal microenvironmental levels of VEGF alone, when delivered through clonal myoblast populations (96.8±10.5%).

The moderate increase in blood flow induced by VEGF alone did not correlate with a measurable increase in collateral artery growth compared to control cells (5.3 and 4.7/leg, respectively), as neither did PDGF-BB alone (4.3/leg; FIG. 5D). VIP cells, instead, caused a doubling in collateral artery number (VIP_{low}=9.7/leg, p<0.01 and VIP_{high}=9.0/leg, p<0.05 for all comparisons; FIG. 5D).

The functional effect of these differences in blood flow improvement and collateral formation were examined. Consistent with the data above, control cells, PDGF-BB and VEGF alone did not improve the percentage of damaged tissue in ischemic muscle (FIG. 5E), although a non-significant trend towards reduction was observed from about 40% to about 30%. It was further demonstrated that both VIP populations reduced damage to less than 15%.

### Example 8

### PDGF-BB Signaling Modulates the Threshold between Normal and Aberrant Angiogenesis

The influence of PDGF-BB signaling on the dose-dependent effects of VEGF were determined by using previously well-characterized clonal populations of transduced myoblasts which homogeneously express defined levels of VEGF (Ozawa, C. R. et al. 2004. J Clin Invest 113, 516-27).

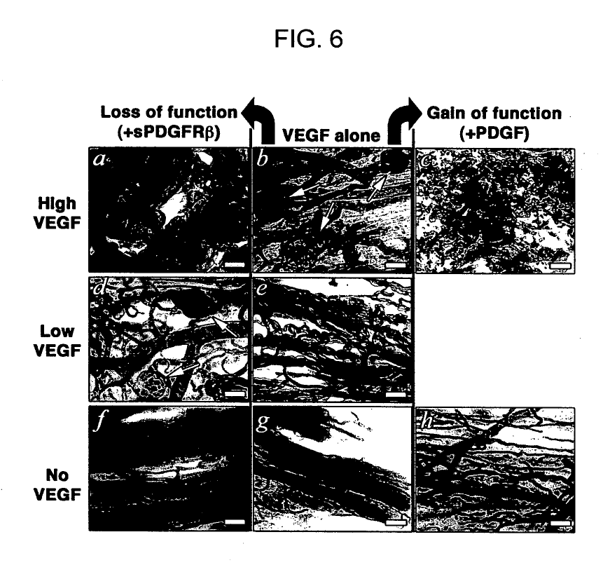

In a gain-of-function experiment, a clone expressing a VEGF level above the threshold (270.9 ng/10⁶ cells/day) was overinfected with the PDGFb retrovirus described above, so that both factors could be delivered with the same high VEGF level being expressed by each cell in the resulting population. Three weeks after implantation, the high VEGF dose alone induced aberrant pre-angiomatous vessels (FIG. 6, Panel B). Co-expression of PDGF-BB (VIP) prevented the appearance of these structures and yielded instead clusters of highly-branched short capillaries (FIG. 6, Panel C), similar to the polyclonal VIP populations. PDGF-BB cells alone did not have an angiogeneic effect (FIG. 6, Panel H).

In a loss-of-function approach, a myoblast population secreting a truncated soluble form of PDGFRβ was generated which, by binding PDGF-BB with high efficiency, prevents it from reaching its receptors, effectively abrogating endogenous PDGFRβ signaling in the implantation site (Duan, D. S., et al. 1991. J Biol Chem 266, 413-8). It was demonstrated that sPDGRβ alone did not have any effect on pre-existing quiescent vasculature compared to control CD8Z cells (FIG. 6, Panels F and G), whereas microenvironmental VEGF levels below the threshold (70.0 ng/10⁶ cells/day) induced only normal, homogeneous capillaries (FIG. 6, Panel E). However, when endogenous PDGF-BB signaling was locally blocked by co-implanting sPDGFRβ cells, the same microenvironmental VEGF level caused the appearance of aberrant structures, similar to those induced by VEGF levels above the threshold (FIG. 6, Panel D). Furthermore, when sPDGFRβ was delivered with above-threshold VEGF levels, aberrant angiogenesis was induced faster and more extensively (FIG. 6, Panel A).

These results demonstrate that the level of PDGF-BB signaling determine the dose at which VEGF induces normal or aberrant angiogenesis.

### Example 9

### Co-Expression of VEGF-A and PDGF-BB by Clonal Myoblast Populations

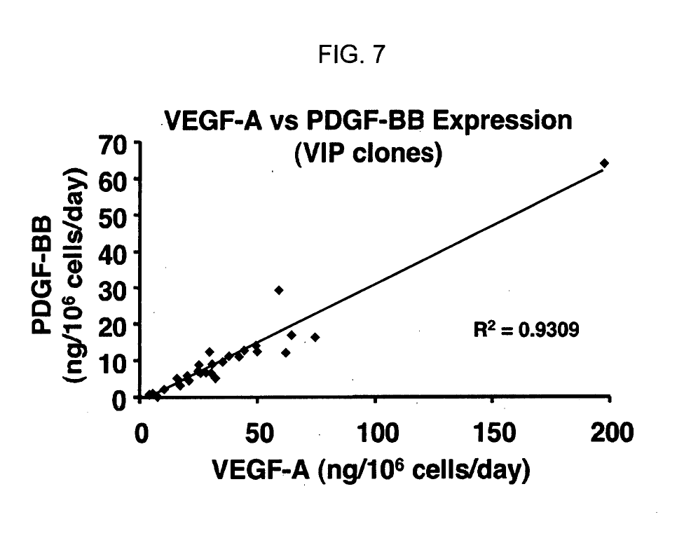

Thirty individual clones were isolated from the polyclonal VIP populations described above and their production of mouse VEGF₁₆₄ and human PDGF-BB quantified by ELISA (VIP clones). The stably transduced individual clones from the VIP constructs co-express mVEGF₁₆₄ and HPDGF-BB as shown in FIG. 7. The presence of both factors in the supernatants was quantified by ELISA techniques. The VEGF dimer production was matched in each clone by a PDGF-BB production of 0.8 to 64 ng/10⁶ cells/day, with a very high correlation coefficient (R²=0.93). In order to account for the different molecular weight of the 2 factors, the nanogram amounts were converted to picomoles which resulted in the ratio of PDGF to VEGF production to be 46±2% in each clone. These results demonstrate that the bicistronic VIP construct allows the automatic matching of the relative level of VEGF and PDGF in each cell in a manner independent of transduction efficiency and integration site variability.

The results reported in the Examples above demonstrate that the threshold between normal and aberrant angiogenesis is not an intrinsic property of VEGF dose alone, but rather is correlated with balance between VEGF and PDGF-BB signaling, and that enhancing vascular maturation by PDGF-BB co-delivery can normalize VEGF-induced angiogenesis. VEGF and PDGF-BB remain extremely localized around the expressing cells, due to a Cell surface Retention Signal (CRS) coded in exon 6, creating a microenvironment at the site of VEGF and PDGF-BB production.

Co-delivery at roughly matched microenvironmental levels by the VIP construct allowed 100% penetrance of PDGF-BB effects on VEGF-induced angiogenesis. The most striking of these were potentiation and normalization. Vessel length density was more than doubled by co-delivery compared to VEGF alone, while PDGF-BB per se caused accumulation of NG2+/SMA-pericytes without new vessels. Furthermore, the induced vessels were normal capillaries of homogeneous size, invested with typical pericytes embedded in the vascular basement membrane. The VEGF-normalizing effects of PDGF-BB in vivo yielded a substantially homogeneous phenotype even if factor production was heterogeneous and high, without a need to precisely control its microenvironmental level distribution in the tissue, in contrast to VEGF alone. The results in FIG. 6, obtained with clonal populations, exclude the possibility that this phenotypic conversion could result from a PDGF-BB-induced selection of a subpopulation expressing homogeneous and optimal levels of VEGF.

Two distinct VIP populations, with an average VEGF expression level of 59 and 108 ng/10⁶ cells/day each significantly improved regional blood flow in ischemic limbs in a model of chronic hindlimb ischemia. This was about twice as much as VEGF alone at a similar level (112 ng/10⁶ cells/day). Furthermore, VIP cells increased collateral formation and reduced ischemia-induced tissue damage, showing that the functionality of VEGF-induced vessels is greatly improved by the normalization and potentiation caused by PDGF-BB co-delivery. Both populations caused similar improvements in all functional parameters analyzed, despite very different average levels of VEGF expression, suggesting that high VEGF expression levels do not need to be reached in order to achieve the beneficial or maximum effect when co-delivered with PDGF-BB.

PDGF-BB and VEGF co-delivery from a single construct to provide selected fixed ratios of PDGF and VEGF polypeptide production can: 1) facilitate the homogenization of the angiogenic phenotype despite very heterogeneous expression levels, since VEGF-PDGF-BB co-delivery from a single construct avoids the need to so precisely control microenvironmental distribution of VEGF; 2) increase safety of use of VEGF to induce antiogenesis, even where VEGF expression levels are relatively high, in view of the ability of PDGF to normalize VEGF-induced angiogenesis by raising the threshold level above which aberrant vessels are induced; 3) provide for improved efficacy of lower, and therefore inherently safer, VEGF levels (e.g., as demonstrated by the observation that blood flow and collateral formation in hindlimb ischemia was doubled by co-delivery compared with VEGF alone, despite a much lower average level of VEGF expression (59 vs. 112 ng/10⁶ cells/day)). In addition, single vector co-delivery of PDGF and VEGF induces vessels that are “mature” “mature”, and facilitates a switch in angiogenic phenotype (from glomeruloid/abnormal to normal).

That which is claimed is:

1. A polynucleotide comprising a nucleic acid sequence of the formula:
P₁-A-Z-B
where
A is a first polynucleotide comprising a first coding sequence for either a VEGF polypeptide or a PDGF polypeptide;
B is a second polynucleotide comprising a second coding sequence wherein, when A encodes a VEGF polypeptide B encodes a PDGF polypeptide or, when A encodes a PDGF polypeptide B encodes a VEGF polypeptide;
P₁, when present, is a first promoter operably positioned to provide for expression of at least A; and
Z is a polynucleotide comprising:
a translation initiation signal “T” operably positioned to facilitate translation of an mRNA encoded by B;
a promoter “P₂”, wherein P₂ is operably positioned to facilitate transcription of an mRNA encoded by B, and wherein P₁, when present facilitates transcription of an mRNA encoded by A; or
a promoter “P₃” and a promoter “P₄” wherein P₃ is operably positioned to provide for transcription of an mRNA encoded by the coding sequence of A and P₄ is operably positioned to provide for transcription of an mRNA encoded by the coding sequence of B, wherein the polynucleotide is double stranded, and wherein the first and second coding sequences are on opposite, complementary strands.

2. The polynucleotide of claim 1, wherein Z is a translation initiation signal, and wherein P₁ is operably positioned to provide for transcription of the first polynucleotide, the translation initiation signal, and the second polynucleotide as a single RNA molecule.

3. The polynucleotide of claim 2, wherein the translation initiation signal is an internal ribosome entry site (IRES).

4. The polynucleotide of claim 3, wherein the first polynucleotide encodes VEGF and the second polynucleotide encodes PDGF.

5. The polynucleotide of claim 1, wherein Z comprises is a second promoter P₂.

6. The polynucleotide of claim 1, wherein P₂ is a promoter that is different from the first promoter P₁.

7. The polynucleotide of claim 5, wherein the first polynucleotide encodes VEGF and the second polynucleotide encodes PDGF.

8. The polynucleotide of claim 1, wherein Z comprises the promoter P₃ and the promoter P₄.

9. A recombinant host cell containing the polynucleotide of claim 1.

10. A population of mammalian cells, wherein at least 5% of mammalian cells in said population contain the polynucleotide of claim 1.

11. A composition for delivery of VEGF and PDGF, wherein the composition comprises:
the polynucleotide according to claim 1 in a pharmaceutically acceptable carrier; or
a pharmaceutically acceptable carrier and a recombinant host cell containing the polynucleotide of claim 1.

12. The composition of claim 11, comprising the polynucleotide or the recombinant host cell, wherein the polynucleotide comprises a bicistronic expression cassette for coordinated co-expression of a VEGF polypeptide and a PDGF polypeptide.

13. The composition of claim 11, comprising the polynucleotide or the recombinant host cell, wherein the polynucleotide comprises a dual expression cassette.

14. The composition of claim 11, comprising the polynucleotide or the recombinant host cell, wherein the first polynucleotide “A” encodes VEGF and the second polynucleotide “B” encodes PDGF.

15. The composition of claim 11, wherein the composition comprises the recombinant host cells seeded on a biologically compatible scaffold.

16. A method of administering a platelet-derived growth factor (PDGF) polypeptide and a vascular endothelial growth factor (VEGF) polypeptide to a subject, the method comprising:
administering the composition of claim 11 to a subject in an amount effective to provide for delivery of PDGF and VEGF in the subject.

17. A method of stimulating angiogenesis and/or vasculogenesis in a subject having a condition that is treatable by stimulating angiogenesis, the method comprising:
administering to a subject the composition of claim 11, wherein said administering of the vector provides for production of VEGF and PDGF to effect stimulation of non-aberrant angiogenesis and/or non-abberrant vasculogenesis in the subject.

18. The method of claim 17, wherein the composition comprises a recombinant host cell.

19. The method of claim 18, wherein the recombinant host cell is an embryonic stem cell, a progenitor cell, or an adult stem cell.

20. The method of claim 17, wherein the composition comprises a population of cells characterized as including at least 60% recombinant cells.
